(19) United States
(12) Patent Application Publication
(10) Pub. No.: US 2023/0277483 A1
(43) Pub. Date: Sep. 7, 2023

(54) COMPOSITIONS AND METHODS TO TREAT INFLAMMATION

(71) Applicant: University of Iowa Research Foundation, Iowa City, IA (US)

(72) Inventors: Liu Hong, Coralville, IA (US); Jeff Banas, Solon, IA (US); Ling Yang, Coralville, IA (US); Min Zhu, Iowa City, IA (US); Yi Shu, Chongqing (CN)

(21) Appl. No.: 18/110,094

(22) Filed: Feb. 15, 2023

Related U.S. Application Data
(60) Provisional application No. 63/310,425, filed on Feb. 15, 2022.

(51) Int. Cl. A61K 31/19; A61K 47/54; A61K 47/64; A61P 1/02; A61P 29/00; A61P 3/04

(52) U.S. Cl. CPC A61K 31/19; A61K 47/54; A61K 47/64; A61P 1/02; A61P 29/00; A61P 3/04

(57) ABSTRACT

A composition comprising an amount of one or more agents comprising a hydroxy(C₁₋₁₀)(COOH) or salt thereof, wherein C₁₋₁₀ can be substituted or form a ring, a dicarboxylic acid, a purine nucleoside or analog thereof, a pyrimidine nucleoside or an analog thereof, or an amino acid or analog thereof, effective to inhibit inflammation, and methods of using the composition, are provided.

### CROSS-REFERENCE TO RELATED APPLICATIONS

This application claims the benefit of the filing date of U.S. application No. 63/310,425, filed on Feb. 15, 2022, the disclosure of which is incorporated by reference herein.

### STATEMENT OF GOVERNMENT RIGHTS

This invention was made with government support under grant R01DE026433 awarded by the National Institutes of Health. The government has certain rights in the invention.

### BACKGROUND

Nearly 50% of American adults have periodontitis, a set of inflammatory diseases that not only cause tooth loss but can also affect systemic health by increasing the risk for atherosclerosis, adverse pregnancy outcomes, rheumatoid arthritis, aspiration pneumonia, and cancer (Eke et al., 2010; Olsen, 2015; Kim et al., 2010; Lin et al., 2014; Pires et al., 2014). The prevalence of periodontitis increases in smokers and in patients with obesity and diabetes (Mathur et al., 2011; D'Aiuto et al., 2008). The sustained chronic inflammatory state of obesity strongly intersects with periodontitis in the context of both pathogenesis and prognosis. Adults with obesity nearly double the prevalence rate of periodontitis compared to non-obese subjects, and obese subjects with periodontitis result in more severe alveolar bone loss.

Current outcomes of periodontitis treatment for moderate to advanced disease and the periodontitis associated with obesity are far from satisfactory. Periodontitis is considered to have a complex etiology acting at multiple levels, including microbial and host contributions (Hajishengallis, 2015); however, the molecular mechanisms underlying the etiology and pathogenesis of periodontitis remain unknown. Oral hygiene, scaling and cleaning, and antibiotics have achieved relative success in arresting the progression of early stage periodontitis that is without systemic disease association, nevertheless, surgical intervention is needed for advanced periodontitis (Smiley et al., 2015; Graziani et al., 2000). The success rate of the current surgical treatment for moderate to advanced periodontitis is only 50% (Lundgren et al., 2001). Thus, effective tools and strategies that improve prevention and therapy outcomes are needed.

### SUMMARY

Streptococcus gordonii (Sg) may modulate interactions between the bacterial community and the host by regulating signaling pathways in host epithelial cells. As disclosed herein, Sg was found to reprogram epithelial cell global transcriptional patterns following Porphyromonas gingivalis (Pg)-induced gingival epithelial cell proliferation. Sg also effectively prevented the invasion of Pg into oral epithelial cells and re-programmed the cells to resist Pg-induced inflammatory transcriptional factors. As also disclosed herein, cell-free supernatants from Sg cultures promoted the growth of health-related bacteria, e.g., Streptococcus sanguinis (Ss), inhibited disease-related bacterial taxa, e.g., Pg, Tannerella forsythia (Tf), and/or Treponema denticola (Td), had global effects on the composition of mouse oral and fecal microbiomes, and downregulated periodontitis and proinflammatory cytokines, e.g., IL-1b, IL-6 and/or IL-9, production in vitro. In vivo administration of Sg products ameliorated adipose inflammation, e.g., IL6, which is associated with obesity-associated systemic chronic glucose intolerance. Components in Sg products that suppress proinflammatory cytokines and upregulate anti-inflammatory periodontitis-associated microRNAs (miRs) were identified, e.g., using UPLC (Ultra Performance Liquid Chromatography). A total of 324 components were present in significantly higher concentrations than in the DMEM control (performed in triplicate, p<0.05). Among them, malic acid (MA), 4-hydroxyphenyllactic acid (HPLA), acadesine (AICAR), uridine (U), citrulline (Cit), and 6-hydroxycaproic acid (HCA) were present at a concentration-related absorbance greater than 100 and are at least 100-fold or greater than in controls. These compounds, individually or in combination, suppress proinflammatory cytokines and upregulate anti-inflammatory periodontitis-associated microRNAs (miRs) in human gingival cells and white adipose tissue (WAT) of obese mice. Thus, these compounds, individually or in any combination, can be used to prevent, inhibit or treat inflammation, pulpitis or periodontitis in patients, e.g., in obese or diabetic patients with periodontitis, and/or improve inflammation and/or metabolism dysregulation, e.g., in obese or diabetic patients.

Thus, the disclosure provides for compositions and methods to prevent, inhibit or treat periodontal and/or adipose tissue inflammation and improve oral microbiome symbiosis. In some embodiments, the compositions may be employed to prevent, inhibit, or treat pulpitis, e.g., using a rinse or gel comprising one of more of the identified compound(s), prevent, inhibit, or treat periodontitis or gingivitis, e.g., via direct injection of the identified compound(s), diabetes, e.g., using a tablet or capsule comprising one or more of the identified compound(s) or a topically applied composition, or osteoarthritis, e.g., via intra-articular injection of one or more of the compound(s). The active compound(s) may be employed as a prebiotic optionally in combination with probiotics, e.g., including probiotic strains such as Streptococcus, Lactobacillus, Lactococcus, or Bifidobacterium.

In one embodiment, the disclosure provides for a composition comprising an amount of one or more agents comprising a hydroxy(alkyl)carboxylic acid, such as a hydroxy(C₁₋₁₀)carboxylic acid, wherein alkyl or C₁₋₁₀ can be substituted, such as 6-hydroxycaproic acid (HCA; aka 6-hydroxyhexanoic acid), a dicarboxylic acid, e.g., (COOH)C₁₋₄(COOH) such as malic acid (MA), a 2-hydroxy carboxylic acid or carboxylate, e.g., (OH)C₁₋₁₀(COOH), wherein C₁₋₁₀ can be substituted or form a ring structure, such as 4-hydroxyphenyl lactic acid (HPLA), a purine nucleoside analogs such as acadesine, a pyrimidine nucleoside, such as uridine, or an analog thereof, or an amino acid such as citrulline or arginine. “Alkyl” as used herein includes cycloalkyl and cycloalkylalkyl groups, Alkyl groups may be substituted to the extent that such substitution makes sense chemically. Typical substituents include, but are not limited to, halo, ═O, ═N—CN, ═N—OR, ═NR, OR, NR₂, SR, ═C, —O—, —N—, —S—, SO₂R, SO₂NR₂, NRSO₂R, NRCONR₂, NRCOOR, NRCOR, CN, COOR, CONR₂, OOCR, COR, and NO₂, wherein each R or R₂ is independently H, C₁-C₈ alkyl, C₂-C₈ heteroalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C₁-C₈ alkyl, C₂-C₈ heteroalkyl, C₁-C₈ acyl, C₂-C₈ heteroacyl, C₆-C₁₀ aryl or C₅-C₁₀ heteroaryl. Alkyl may also be substituted by C₁-C₈ acyl, C₂-C₈ heteroacyl, C₆-C₁₀ aryl or C₅-C₁₀ heteroaryl, each of which can be substituted by the substituents that are appropriate for the particular group.

In one embodiment, the disclosure provides for a composition comprising an amount of one or more agents comprising 6-hydroxycaproic acid (HCA), malic acid (MA), 4-hydroxyphenyl lactic acid (HPLA), acadesine, uridine, or citrulline, or any combination thereof, e.g., in an amount effective to inhibit one or more of Porphyromonas gingivitis (Pg), Tannerella forsythia (Tf), and/or Treponema denticola (Td), or to prevent, inhibit or treat inflammation, periodontitis, pulpitis, gingivitis, diabetes, or osteoarthritis. In one embodiment, the composition is a paste for administration to the teeth or gums or skin. In one embodiment, the composition is a gel, e.g., for administration to the teeth, gums or skin. In one embodiment, the composition is suitable for injection. In one embodiment, the composition is suitable for topical application. In one embodiment, the composition is a beverage or a foodstuff. In one embodiment, the agent is linked to a targeting molecule. In one embodiment, the targeting molecule targets dental plaque. In one embodiment, the composition comprises HCA. In one embodiment, the composition comprises two or more of HCA, malic acid, HPLA, acadesine, uridine, or citrulline.

Also provided is a method to prevent, inhibit or treat inflammation in a mammal, comprising: administering to the mammal a composition comprising an effective amount of one or more of 6-hydroxycaproic acid (HCA), mail acid, 4-hydroxyphenyl lactic acid, acadesine, uridine, or citrulline. In one embodiment, the mammal is a human. In one embodiment, the mammal has osteoarthritis. In one embodiment, the mammal is obese. In one embodiment, the composition is systemically administered. In one embodiment, the administration is oral administration. In one embodiment, the composition is locally administered. In one embodiment, the administration is intra-articular administration. In one embodiment, the administration is to the gums, e.g., via application of a gel or injection. In one embodiment, the composition is injected. In one embodiment, the composition is a sustained release formulation. In one embodiment, the mammal has periodontitis, gingivitis or pulpitis. In one embodiment, the composition is a paste or gel.

### BRIEF DESCRIPTION OF THE FIGURES

FIGS. 1A-1B. Sg supernatant inhibits proinflammatory cytokines and promotes anti-inflammatory miRs. A: relative fold changes of the transcripts of IL-16, 6, and 8 in RAW 264.7 after treatment with Sg and/or Pg supernatant at the same OD600 value. B: relative fold changes of the transcripts of miR-146, 17, and 200c in mouse macrophages 24 hours after treatment with Sg supernatant at 5%. Performed in duplicate.

FIGS. 2A-2D. Sg metabolites modulate proinflammatory cytokines in human macrophages and HGF. A-B: relative fold changes of the transcripts of IL-16, 6, and 8 in human MDM and HGF treated with Sg metabolites at 1 and 5% after Pg-LPS challenge. Performed in triplicate. *: p<0.05 vs LPS. C-D: Representative images (C) and signal intensities (D) obtained with RayBio C-Series Antibody Arrays. The membranes were probed with the supernatant of HGF pretreated with Sg metabolites or a control after challenge with LPS. Performed in triplicate. *:0.05 vs LPS. Pos: positive controls.

FIGS. 3A-3D. Sg metabolites promoted Sg and So proliferation and inhibited pathogenic bacterial proliferation and colonization. A-B: Normalized fold change of So and Sg (A), and Td, Tf and Pg (B) after treatment with Sg metabolite at 5%. C-D: Representative Live/Dead images (C) and attachment area (D) of Pg after different treatments. Performed in triplicate. *: p<0.05 vs LPS.

FIGS. 4A-4B. L-Norleucine modulates proinflammatory cytokines in macrophages and HGF. A-B: Relative fold changes of IL-16, 6, and 8 of macrophages (A) and HGF (B) treated with L-Norleucine (NL) at 1 and 5 μM after Pg-LPS challenge. Performed in duplicate.

FIGS. 5A-5G. Sg SCS promotes Sg and Ss proliferation and inhibits pathogenic bacterial proliferation and colonization. A-E: Proliferation of Sg (A), Ss (B), Pg (C), Tf (D), and Td (E) measured by OD600 after treatment with 1 or 5% v/v DMEM and Sg SCS compared to controls, and performed in triplicate. *: p<0.05 vs. DMEM; F-G: Representative Live/Dead images (F) and attachment area (G) of Pg after different treatments. Data are presented as means±SEM, * indicates statistical significance compared to the control (DMEM) group as determined by one-way ANOVA test, p<0.05.

FIGS. 6A-6C. Comparison of Streptococcus species on regulation of proinflammatory cytokines. A-C: relative fold changes of the transcripts of IL-1β (A), 6 (B), and 8 (C) in mouse macrophages treated with SCS from different Streptococcus species after Pg-LPS challenge, and performed in triplicate. Data are presented as means±SEM, * indicates statistical significance compared to the control (LPS) group as determined by one-way ANOVA test, p<0.05 vs LPS.

FIGS. 7A-7C. Sg SCS modulates proinflammatory cytokines in human macrophages and HGF. A-B: relative fold changes of the transcripts of IL-1β, 6, and 8 in human MDM and HGF treated with Sg SCS at 1, 5, and 10% after Pg-LPS challenge, and performed in triplicate. *: p<0.05 vs LPS. C: IL-6 levels in the supernatants of HGF treated with Sg SCS measured by ELISA. Data are presented as means±SEM, * indicates statistical significance compared to the control (LPS) group as determined by one-way ANOVA test, p<0.05 vs LPS.

FIGS. 8A-8G. Sg SCS improves obesity-associated metabolic dysfunction. A. Body weight, B. 16-hour fasting glucose level, C. Glucose tolerance test (GTT) and AUC analysis (D) in mice on a HFD (on diet for 16 weeks) subjected to Sg treatments, n=4-5 mice/group. E. Representative light microscopy and H&E images (10×) in eWAT from mice in (A). F. Level of mRNAs encoding tested genes in eWAT from mice in A. G. Representative immunofluorescent staining with BODIPY (lipid) and F4/80 in the liver of mice in A. Scale bar: 10 μm. Data are presented as means±SEM, * indicates statistical significance compared to the control (vehicle) group as determined by Student's t test, p<0.05.

FIGS. 9A-9C. Key Sg metabolites modulate proinflammatory cytokines in human macrophages and HFG. A-B: Relative transcript fold changes of IL-1β, 6 and 8 in HGF (A) and IL-6 transcript in MDM (B) treated with AJCAR, MA, HCA, and HPLA at 0.1 μg after Pg-LPS challenge; C: IL-6 protein levels in supernatants of MDM treated with the Sg metabolites, and performed in duplicate.

FIGS. 10A-10B. Sg metabolites reduce proinflammatory signature of the eWAT in obese mice. A. Levels of mRNAs encoding for tested genes and B. Secreted levels of tested cytokine in eWAT explant freshly isolated from mice on a HFD for 16 weeks, followed by treatment of indicated drugs. (n=4 mice/group). All data are presented as means±SEM. * indicates statistical significance compared to the control group as treatment by ANOVA, p<0.05.

FIGS. 11A-11B. Sg metabolites upregulate anti-inflammatory miRs in human macrophages and HGF. A-B: Relative transcript changes of miR-200c, 146a, and 21 in HGF (A) and human MDM (B) treated with AICAR, MA, HCA, and HPLA 24 hours after Pg-LPS challenge; performed in duplicate.

FIG. 12. miRNA-200c changes.

FIGS. 13A-13B. A) Graph of liver transcripts in presence or absence of HCA. B) Graph of liver transcripts for FasN, HsI, Strebp1c, Atg1 and Cd3b in presence or absence of HCA.

FIGS. 14A-14G. A) Graph of weight in animals over time on a regular diet (RD) or high fat diet (HFD) with or without HCA. B) Percent fat, lean and body fluid in animals on a HFD with or without HCA. C) Activity in light or dark without or without HCA. D) RER/kg in light or dark without or without HCA. E) Tissue sections from eWAT, iWAT or liver from animals with or without HCA. F) Serum ALT for animals on a HFD with or without HCA. G) Serum FFA for animals on a HFD with or without HCA.

FIGS. 15A-15C. A) Fasting glucose for animals fed RD or HFD with or without HCA. B) Glucose over time after insulin for animals fed RD or HFD with or without HCA and ITT AUC. C) eWAT pAkt/Akt for animals fed RD or HFD with or without HCA.

FIGS. 16A-16E. A) Expression profiles. B) eWATmRNA and various genes for animals fed HFD with or without HCA. C) ILbeta expression in animals fed HFD with or without HCA. D) IL-6 expression in animals fed HFD with or without HCA. E) Leptin expression in animals fed HFD with or without HCA.

FIG. 17. IL6 change in control, HCA, TNF or TNF and HCA treatments.

FIGS. 18A-18D. HCA enhances commensal bacterial proliferation and inhibits proliferation and attachment of periodontal pathogenic bacteria. A-C: Growth profiles of S. mitis (4), S. oralis (B), and T. denticola (C) after treatment with HCA at 0.1 μM. D: P. gingivalis attachment to cultured plates after treatment with HCA 0.01 and 0.05 μM as measured by optical density. *: p<0.05 vs control; performed in triplicate.

FIGS. 19A-19B. HCA reduces Pg-LPS-mediated inflammation in human macrophages and HGF. Left: Viability (%, measured using MTT) in HGF (A) and human MDM (B) 24 and 48 hours after treatment with HCA at 0.1, 1, 10, and 50 μM. Levels of transcripts (Middle, measured using qRT-PCT) and proteins (Right column, measured using ELISA) of proinflammatory cytokines in HGF (A) and MDM (B) treated with HCA at 1 μM after Pg-LPS challenge. All data are presented as means±SEM. *indicates statistical significance compared to control, # indicates statistical significance compared to LPS as determined by one-way ANOVA, followed by Post-Hoc Tukey test, p<0.05. N=3;

FIGS. 20A-20C. HCA modulates periodontal inflammation and affects oral and fecal composition of microbiomes in vivo. A-B: Gingival IL-1β transcript (A) and serum IL-1β measured with ELISA (B) of mouse gingival injection with Pg-LPS (10 μg) twice a week alone or in conjunction with 100 μl of IP HCA (0.5 μM) three times weekly; *indicates statistical significance compared to untreated control, # indicates statistical significance compared to LPS, p<0.05. N=3; C: Species distributions in the oral and fecal microbiomes of obese mice with 100 μl of IP HCA (0.2 and 0.5 μM) three times weekly.

FIG. 21. A heatmap of FFA related receptors in WAT of obese mice after HCA treatment.

### DETAILED DESCRIPTION

Nearly half of American adults have periodontitis, a chronic inflammatory disease leading to tooth loss and closely linked to many systemic diseases. While a disrupted balance in the subgingival microbiome sets the stage for initiation of the disease, the imbalance and dysregulation of host inflammatory responses exaggerates the progression of the periodontitis. Currently available approaches have limited effectiveness in efficiently preventing or treating periodontitis, especially when it associates with systemic diseases. Health-related commensal bacteria are generally considered to compete with the periodontopathogenic bacteria and represent a barrier to their invasion. Accumulated evidence further indicates that the commensal bacteria might potently modulate inflammatory responses of oral cells that in turn mitigate the inflammation and progression of periodontitis. Therefore, there is need to determine the functions and underlying mechanism(s) of specific metabolites produced by commensal bacteria that promote homeostasis in the oral microbiome and host inflammatory response in order to develop commensal bacteria-based approaches for periodontitis prevention and treatment.

### Definitions

Unless defined otherwise, all technical and scientific terms used herein have the same meaning as is commonly understood by one of skill in the art to which the claimed subject matter belongs.

It is to be understood that the foregoing general description and the following detailed description are exemplary and explanatory only and are not restrictive of any subject matter claimed. In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated.

As used herein, the term “isolated” refers to in vitro preparation and/or isolation of a molecule so that it is not associated with in vivo substances, or is substantially purified from in vitro substances.

As used herein, “about” means±5% of the indicated range, value, sequence, or structure, unless otherwise indicated.

It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously.

As used herein, the term “formulation” or the term “formulations” may include a plurality of ingredients including, for example, one or more of malic acid (MA), 4-hydroxyphenyllactic acid (HPLA), acadesine (AICAR), uridine (U), citrulline (Cit), and/or 6-hydroxycaproic acid (HCA). Such formulations may include other ingredients such as carriers, excipients, solvents, flavors, or any other ingredients as further described herein. For common excipients and carriers, see Remington's Pharmaceutical Sciences, infra, listing various excipients, diluents, additives, carriers, lubricants, and the like.

As used herein, the term “animal” includes any living organism characterized by voluntary movement.

As used herein, the term “subject” may include a mammal such as a human, unless specified otherwise.

As used herein, the term “%” or “wt. %” is a percent by weight value based on a total weight (taken as 100 wt. %) of the formulation with a specific set of ingredients or all of its constituent ingredients accounted for, such as listed ingredients (active and/or inactive), excipients (if any), carriers (if any), or other active ingredients (if any), unless indicated otherwise.

As used herein, the term “organic” may include ingredients that may be farmed or grown without the use of certain pesticides, antibiotics and/or genetically altered or genetically modified plants, seeds, or organisms.

As used herein, the term “non-organic” may include ingredients that may be farmed or grown, optionally with the use of pesticides, antibiotics and/or genetically altered or genetically modified plants, seeds, or organisms.

As used herein, the term “treatment” may include care provided to improve a condition or one or more symptoms thereof or alleviate some discomfort or undesirable consequence of the condition such as a chronic disease or condition, or even an acute disease or condition or illness, etc.

As used herein, the term “symptomatic relief” may include control or alleviation of symptoms and/or side effects obtained with the aid, application or ingestion of one or more embodiments of the disclosed compositions.

### Compositions and Formulations

According to one or more illustrative embodiments, formulation(s) in connection with the present disclosure may include one or more of the compound(s) and optionally other constituents described herein.

Any one or more of the formulation(s) or embodiment(s) thereof described herein may have varying ingredient contents. Thus, for example, the amount of each ingredient may fluctuate within a certain range (but not limited to): about ±1%, up to about ±10%, as disclosed herein. The various embodiments may also include well-known excipients, carriers, fillers, pigments, colorants, preservatives, diluents, solvents, and emulsifiers suitable for use in formulations. Such embodiments may optionally include active ingredients in various forms, including, but not limited to, gels, pastes liquids, powders, syrups, shakes, tablets, capsules, e.g., gelatin capsules, concentrates, pills, emulsions, and the like.

In the one or more embodiments, one or more of malic acid (MA), 4-hydroxyphenyllactic acid (HPLA), acadesine (AICAR), uridine (U), citrulline (Cit), and/or 6-hydroxycaproic acid (HCA) optionally with any pharmaceutically acceptable carrier may be provided in, for example, a liquid, e.g., an oral rinse, paste, e.g., toothpaste, gel, e.g., an oral gel, powder, e.g., added to a base powder or base liquid, a gum, or a food product, thereby providing a mixture.

In one embodiment of a composition, one or more of malic acid (MA), 4-hydroxyphenyllactic acid (HPLA), acadesine (AICAR), uridine (U), citrulline (Cit), and 6-hydroxycaproic acid (HCA) may range from about 0.01 μM to about 10 μM.

In one embodiment of a composition, one or more of malic acid (MA), 4-hydroxyphenyllactic acid (HPLA), acadesine (AICAR), uridine (U), citrulline (Cit), and 6-hydroxycaproic acid (HCA) may range from about 0.01 mM to about 10 mM.

In one embodiment, the HCA may range from about 0.01 μM to about 10 μM or about 0.01 mM to about 100 mM.

In one embodiment, the MA may range from about 0.01 μM to about 10 μM.

In one embodiment, the HPLA may range from about 0.01 μM to about 10 μM or about 0.01 mM to about 100 mM.

In one embodiment, the AICAR may range from about 0.01 μM to about 10 μM or about 0.01 mM to about 100 mM.

In one embodiment, the uridine may range from about 0.01 μM to about 10 μM or about 0.01 mM to about 100 mM.

In one embodiment, the Cit may range from about 0.01 μM to about 10 μM or about 0.01 mM to about 100 mM.

In one embodiment of a composition, one or more of malic acid (MA), 4-hydroxyphenyllactic acid (HPLA), acadesine (AICAR), uridine (U), citrulline (Cit), and 6-hydroxycaproic acid (HCA) may range from about 0.001 μM to about 100 μM.

In one embodiment of a composition, one or more of malic acid (MA), 4-hydroxyphenyllactic acid (HPLA), acadesine (AICAR), uridine (U), citrulline (Cit), and 6-hydroxycaproic acid (HCA) may range from about 0.01 mM to about 100 mM

In one embodiment, the HCA may range from about 0.01 μM to about 10 μM, 0.1 μM to about 100 μM, 0.1 μM to about 1000 μM, 0.01 mM to about 1 mM, 0.1 mM to about 10 mM, or 1 mM to about 100 mM.

In one embodiment, the MA may range from about 0.01 μM to about 10 μM, 0.1 μM to about 100 μM, 0.1 μM to about 1000 μM, 0.01 mM to about 1 mM, 0.1 mM to about 10 mM, or 1 mM to about 100 mM.

In one embodiment, the HPLA may range from about 0.01 μM to about 10 μM, 0.1 μM to about 100 μM, 0.1 μM to about 1000 μM, 0.01 mM to about 1 mM, 0.1 mM to about 10 mM, or 1 mM to about 100 mM.

In one embodiment, the AICAR may range from about 0.01 μM to about 10 μM, 0.1 μM to about 100 μM, 0.1 μM to about 1000 μM, 0.01 mM to about 1 mM, 0.1 mM to about 10 mM, or 1 mM to about 100 mM.

In one embodiment, the uridine may range from about 0.01 μM to about 10 μM, 0.1 μM to about 100 μM, 0.1 μM to about 1000 μM, 0.01 mM to about 1 mM, 0.1 mM to about 10 mM, or 1 mM to about 100 mM.

In one embodiment, the Cit may range from about 0.01 μM to about 10 μM, 0.1 μM to about 100 μM, 0.1 μM to about 1000 μM, 0.01 mM to about 1 mM, 0.1 mM to about 10 mM, or 1 mM to about 100 mM.

In one embodiment of a composition, one or more of malic acid (MA), 4-hydroxyphenyllactic acid (HPLA), acadesine (AICAR), uridine (U), citrulline (Cit), and 6-hydroxycaproic acid (HCA) may range from about 10/to about 20%, from about 2% to about 8%, from about 23% to about 29% or from about 52% to about 58% of the total weight of the composition.

In one embodiment of a composition, one or more of malic acid (MA), 4-hydroxyphenyllactic acid (HPLA), acadesine (AICAR), uridine (U), citrulline (Cit), and 6-hydroxycaproic acid (HCA) may range from about 1% to about 2%, from about 0.2% to about 0.8%, from about 2% to about 3% or from about 5% to about 10% of the total weight of the composition.

In one embodiment, the HCA may range from about 12% to about 18%, 2.5% to about 8%, 25% to about 29% or from about 52% to about 56% of the total weight of the composition.

In one embodiment, the MA may range from about 12% to about 18%, 2.5% to about 8%, 25% to about 29% or from about 52% to about 56% of the total weight of the composition.

In one embodiment, the HPLA may range from about 12% to about 18%, 2.5% to about 8%, 25% to about 29% or from about 52% to about 56% of the total weight of the composition.

In one embodiment, the AICAR may range from about 12% to about 18%, 2.5% to about 8%, 25% to about 29% or from about 52% to about 56% of the total weight of the composition.

In one embodiment, the uridine may range from about 12% to about 18%, 2.5% to about 8%, 25% to about 29% or from about 52% to about 56% of the total weight of the composition.

In one embodiment, the Cit may range from about 12% to about 18%, 2.5% to about 8%, 25% to about 29% or from about 52% to about 56% of the total weight of the composition.

In one embodiment, the HCA may range from about 1% to about 2%, 0.25% to about 0.8%, 2.5% to about 3% or from about 5% to about 8% of the total weight of the composition.

In one embodiment, the MA may range from about 1% to about 2%, 0.25% to about 0.8%, 2.5% to about 3% or from about 5% to about 8% of the total weight of the composition.

In one embodiment, the HPLA may range from about 1% to about 2%, 0.25% to about 0.8%, 2.5% to about 3% or from about 5% to about 8% of the total weight of the composition.

In one embodiment, the AICAR may range from about 1% to about 2%, 0.25% to about 0.8%, 2.5% to about 3% or from about 5% to about 8% of the total weight of the composition.

In one embodiment, the uridine may range from about 1% to about 2%, 0.25% to about 0.8%, 2.5% to about 3% or from about 5% to about 8% of the total weight of the composition.

In one embodiment, the Cit may range from about 1% to about 2%, 0.25% to about 0.8%, 2.5% to about 3% or from about 5% to about 8% of the total weight of the composition.

The compositions can be formulated in any suitable product form. Such product forms include but are not limited to a solid, a gel, a paste or a solution, e.g., a liquid, a dispersion, an emulsion, or a powder. The present compositions may include a carrier. A useful carrier is one that is acceptable for ingestion. Useful carriers may include, but are not limited to, one or more aqueous systems, oils such as vegetable or mineral oils, water, pharmaceutically acceptable carrier, non-water beverages, gum paste, or food stuff. The compositions may be conveniently incorporated into a variety of liquids, e.g., a beverage, or solid compositions, e.g., an article of food, a tablet or pill, such as a capsule.

The compositions may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, which may be a diluent or excipient that is compatible with the other ingredients of the formulation and not deleterious to the user. Thus, the compositions may be administered in combination with a pharmaceutically acceptable carrier. The active ingredients in such formulations may comprise from 1% by weight to 99% by weight, or alternatively, 0.1% by weight to 99.9% by weight.

In one embodiment, the composition is optionally provided in liquid form, gel form, paste form, or powder form.

In one embodiment, the composition comprises HCA in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, 20%, ±21%, 22%, ±23%, 24%, and ±25% for each of the mg values, respectively.

In one embodiment, the composition comprises HPLA in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, or 500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

In one embodiment, the composition comprises Cit or AICAR in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg, 1600 mg, 1700 mg, 1800 mg, 1900 mg, 2000 mg, 2500 mg, 3000 mg, 3500 mg, 4000 mg, 4500 mg, or 5000 mg and being within about at least one of or just one of ±1%, ±2%, ±3%, ±4%, ±5%, ±6%, 7%, 8%, 9%, 10%, 11%, 12%, ±13%, ±14%, ±15%, ±16%, ±17%, ±18%, ±19%, ±20%, ±21%, ±22%, ±23%, 24%, and ±25% for each of the mg values, respectively.

In one embodiment, the composition comprises uridine or MA in an amount of about 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg, 1600 mg, 1700 mg, 1800 mg, 1900 mg, 2000 mg or 2500 mg and being within about at least one of or just one of ±1%, ±2%, ±3%, ±4%, ±5%, 6%, 7%, 8%, 9%, 10%, 11%, 12%, 13%, 14%, 15%, 16%, ±17%, ±18%, ±19%, ±20%, ±21%, ±22%, ±23%, 24%, and ±25% for each of the mg values, respectively.

In one embodiment, the composition comprises uridine or Cit in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, or 1500 mg, and being within about at least one of or just one of ±1%, ±2%, ±3%, ±4%, ±5%, ±6%, ±7%, ±8%, ±9%4, ±10%, 11%, 12%, 13%, 14%, 15%, 16%, 17%, 18%, 19%, ±20%, ±21%, ±22%, ±23%, 24%, and ±25% for each of the mg values, respectively.

In one embodiment, the composition comprises HCA in an amount of about 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, or 1500 mg, and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

In one embodiment, the composition comprises HPLA in an amount of about 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, or 1000 mg and being within about at least one of or just one of ±1%, ±2%, ±3%, ±4%, ±5%, ±6%, ±7%, ±8%, ±⁹⁰%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

In some embodiments, the concentration of HCA or HPLA in a liquid formulation is from about 1% to about 10% by total weight. In some cases, the concentration of HCA or HPLA is from about 0.5% to about 15%, about 1% to about 10%, about 2% to about 8%, or about 3% to about 6% by total weight.

In some embodiments, the concentration of AICAR or Cit in a liquid formulation is from about 0.1% to about 1% by total weight. In some instances, the concentration of AICAR or Cit is from about 0.5% to about 10%, about 1% to about 8%, about 2% to about 7%, or about 3% to about 6% by total weight.

In some embodiments, concentration of MA or U in a liquid formulation is from about 0.3% to about 0.6% by total weight.

In some embodiments, the concentration of MA or U in a liquid formulation is from about 0.5% to about 5% by total weight. In some instances, the concentration of MA or U is from about 1% to about 30%, about 2% to about 20%, about 4% to about 16%, or about 6% to about 12% by total weight.

In some embodiments, one or more formulations described herein may be used as a dietary supplement. The formulations may comprise either the above listed ingredients, its active compounds, or said ingredients and active compounds plus one or more nutraceutically acceptable carriers. In addition to the ingredients discussed above, methods of determining active ingredients and screening for activity in the formulations described herein can be carried out according to methods known to those of skill in the art, and according to methods described in the examples herein. Formulations described herein may be mixed with nutraceutically acceptable carriers known to those of skill in the art, and administered according to methods known to those of skill in the art including: oral administration in the form of juice or milk or other beverage, powders, tablets, suspension, emulsifiers, capsules, granules, suspensions, spirits, or syrups.

In addition, well-known excipients in the form of solid or liquid maybe used. The several examples of excipients used to administer the dosage forms may include for powders for oral administration: lactose, crystalline cellulose, starch, dextrin, calcium phosphate, calcium carbonate, synthetic and natural aluminum dioxide, magnesium oxide, dried aluminum hydroxide, magnesium stearate, and/or sodium bicarbonate; Excipients in liquids may include water, glycerin, propylene glycol, sweet-taste syrup, ethanol, fatty oil, ethylene glycol, polyethylene glycol, or sorbitol.

In one embodiment, the ratio of HPLA to HCA in a composition is about 3:1, 2:1, 0.5:2, 1:1, 1:2 or 1:3.

In one embodiment, the ratio of MA or AICAR to HPLA or HCA in a composition is about 1:2, 1:3, 1:4 or 1:6.

In one embodiment, the ratio of Cit or U to HPLA or HCA in a composition is 1:6, 1:4, 1:10, or 1:1.

### Exemplary Delivery Vehicles

Delivery vehicles for the compound(s) in the compositions include, for example, naturally occurring polymers, microparticles, nanoparticles, liposomes, and other macromolecular complexes capable of mediating delivery of a nucleic acid to a host cell. Vehicles can also comprise other components or functionalities that further modulate, or that otherwise provide beneficial properties.

In one embodiment, the delivery vehicle is a naturally occurring polymer, e.g., formed of materials including but not limited to albumin, collagen, fibrin, alginate, extracellular matrix (ECM), e.g., xenogeneic ECM, hyaluronan (hyaluronic acid), chitosan, gelatin, keratin, potato starch hydrolyzed for use in electrophoresis, or agar-agar (agarose). In one embodiment, the delivery vehicle comprises a hydrogel. In one embodiment, the composition comprises a naturally occurring polymer. For example, the compound(s) may be in nanoparticles or microparticles. Table 1 provides exemplary materials for delivery vehicles that are formed of naturally occurring polymers and materials for particles.

TABLE 1

| Particle class | Materials |
|---|---|
| Natural materials or | Chitosan |
| derivatives | Dextran |
| | Gelatine |
| | Albumin |
| | Alginates |
| | Liposomes |
| | Starch |
| Polymer carriers | Polylactic acid |
| | Poly(cyano)acrylates |
| | Polyethyleneimine |
| | Block copolymers |
| | Polycaprolactone |

An exemplary polycaprolactone is methoxy poly(ethylene glycol)/poly(epsilon caprolactone). An exemplary poly lactic acid is poly(D,L-lactic-co-glycolic)acid (PLGA).

Some examples of materials include but are not limited to agar acrylic polymers, polyacrylic acid, poly acryl methacrylate, gelatin, poly(lactic acid), pectin(poly glycolic acid), cellulose derivatives, cellulose acetate phthalate, nitrate, ethyl cellulose, hydroxyl ethyl cellulose, hydroxypropylcellulose, hydroxyl propyl methyl cellulose, hydroxypropylmethylcellulose phthalate, methyl cellulose, sodium carboxymethylcellulose, poly(ortho esters), polyurethanes, poly(ethylene glycol), poly(ethylene vinyl acetate), polydimethylsiloxane, poly(vinyl acetate phthalate), polyvinyl alcohol, polyvinyl pyrrollidone, and shellac. Soluble starch and its derivatives for particle preparation include amylodextrin, amylopectin and carboxy methyl starch.

In one embodiment, the delivery vehicle is biodegradable. Examples of biodegradable polymers include synthetic polymers, e.g., polyesters, poly(ortho esters), polyanhydrides, or polyphosphazenes; natural polymers including proteins (e.g., collagen, gelatin, and albumin), or polysaccharides (e.g., starch, dextran, hyaluronic acid, and chitosan). For instance, a biocompatible polymer includes poly (lactic) acid (PLA), poly (glycolic acid) (PLGA). Natural polymers that may be employed in particles (or as the delivery vehicle) include but are not limited to albumin, chitin, starch, collagen, chitosan, dextrin, gelatin, hyaluronic acid, dextran, fibrinogen, alginic acid, casein, fibrin, and polyanhydrides.

In one embodiment, the delivery vehicle is a gel or hydrogel. Hydrogels can be classified as those with chemically crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible, biodegradable, support cellular activities, and include proteins like fibrin, collagen and gelatin, and polysaccharides like starch, alginate and agarose.

In one embodiment, a vehicle comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(p-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines. A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam): or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed. In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers. DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

### Formulations and Dosages

One or more suitable unit dosage forms comprising the compound(s), which may optionally be formulated for sustained release, can be administered by a variety of routes including local, e.g., oral or topical, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the compound(s) with liquid carriers, solid matrices, semi-solid carriers, finely divided solid carriers or combinations thereof, and then, if necessary, introducing or shaping the product into the desired delivery system.

The amount of the compound(s) administered to achieve a particular outcome will vary depending on various factors including, but not limited to the condition, patient specific parameters, e.g., height, weight and age, and whether prevention or treatment, is to be achieved.

The compound(s) may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The compound(s) may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The compound(s) can be provided in a dosage form containing an amount effective in one or multiple doses. The therapeutic nucleic acid may be administered in dosages of at least about 0.0001 mg/kg to about 20 mg/kg, of at least about 0.001 mg/kg to about 0.5 mg/kg, at least about 0.01 mg/kg to about 0.25 mg/kg, at least about 0.1 mg/kg to about 0.25 mg/kg of body weight, about 0.1 mg/kg to about 0.5 mg/kg, about 0.5 mg/kg to about 2 mg/kg, about 1 mg/kg to about 5 mg/kg, about 5 mg/kg to about 10 mg/kg, or about 10 mg/kg to about 20 mg/kg although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of therapeutic nucleic acid can be administered.

Pharmaceutical formulations containing the compound(s) can be prepared by procedures known in the art using well known and readily available ingredients. For example, the active agent can be formulated with common excipients, diluents, or carriers, and formed into tablets, capsules, suspensions, powders, and the like. The compound(s) may be formulated as particles or complexes, or can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the composition comprising the compound(s) may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

The local delivery of the composition can also be by a variety of techniques which administer the composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

Thus, the compound(s) can be formulated as pharmaceutical compositions and administered to a mammalian host, such as a human patient in a variety of forms adapted to the chosen route of administration, e.g., orally or parenterally, by intravenous, intramuscular, topical, local, or subcutaneous routes. In one embodiment, the composition having the compound(s) is administered prophylactically.

In one embodiment, the compound(s) may be administered by infusion or injection. Solutions of the compound(s) or its salts, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in complexes, liposomes, nanoparticles or microparticles. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, inclusion of isotonic agents, for example, sugars, buffers or sodium chloride is envisioned. Prolonged absorption of the injectable compositions can be brought about by the use in the compositions of agents delaying absorption, for example, microparticles, or aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compound(s) can be determined by comparing their in vitro activity and in vivo activity in animal models thereof. Methods for the extrapolation of effective dosages in mice, and other animals, to humans are known to the art; for example, see U.S. Pat. No. 4,938,949.

The concentration of the compound(s) in a liquid, gel or paste composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the compound(s) for use alone or with other agents will vary with the route of administration, the nature of the condition being treated and the age and condition of the patient and will be ultimately at the discretion of the attendant physician or clinician.

The compound(s) may be conveniently administered in unit dosage form; for example, containing 5 to 1000 mg, conveniently 10 to 750 mg, or conveniently 50 to 500 mg of active ingredient per unit dosage form.

In general, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

### Exemplary Formulations for Oral Delivery

An oral composition may be a toothpaste or a dentifrice, a mouthwash or a mouth rinse, a topical oral gel, and a denture cleanser. A composition may be employed in a method to improve oral health comprising applying an effective amount of the one or more compounds to a subject in need thereof. As used herein, the term “dentifrice” means paste, gel, or liquid formulations unless otherwise specified. The dentifrice composition can be in any desired form such as deep striped, surface striped, multi-layered, having the gel surrounding the paste, or any combination thereof. Alternatively, the oral composition may be dual phase dispensed from a separated compartment dispenser.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

The compositions are intended for topical use in the mouth and so salts should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al, and U.S. Pat. No. 3,678,154, to Widder et al., each of which are incorporated herein by reference. Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts such as where the fluoride is covalently bound to another atom, e.g., as in sodium monofluorophosphate, rather than merely ionically bound, e.g., as in sodium fluoride are envisioned.

The composition may in some embodiments contain anionic surfactants, e.g., water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate: higher alkyl sulfates, such as sodium lauryl sulfate; higher alkyl-ether sulfates, e.g., sodium laureth-2 sulfate, higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C.sub.6-30 alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another embodiment, cationic surfactants may be employed, e.g., derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

In still another embodiment amphoteric surfactants can be used. Suitable amphoteric surfactants, without limitation, are derivatives of C.sub.8-20 aliphatic secondary and tertiary amines having an anionic group such as carboxylate, sulfate, sulfonate, phosphate or phosphonate. A suitable example is cocoamidopropyl betaine. One or more surfactants are optionally present in a total amount of 0.01 weight % to 10 weight %, for example, from 0.05 weight % to 5 weight % or from 0.1 weight % to 2 weight % by total weight of the composition.

The surfactant or mixtures of compatible surfactants can be present in the compositions in 0.1% to 5%, in another embodiment 0.3% to 3% and in another embodiment 0.5% to 2% by weight of the total composition.

The oral care compositions of the invention may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

The oral care compositions of the invention also may include one or more chelating agents able to complex calcium found in the cell walls of the bacteria. Binding of this calcium weakens the bacterial cell wall and augments bacterial lysis.

Another group of agents suitable for use as chelating or anti-calculus agents are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.5 wt. % pyrophosphate ions, 0.9-3 wt. %. The pyrophosphates also contribute to preservation of the compositions by lowering water activity.

The oral care compositions of the invention also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, a thickening material may be included to provide a desirable consistency or to stabilize or enhance the performance of the formulation. In certain embodiments, the thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total The oral care compositions may also optionally include one or more enzymes. Useful enzymes include any of the available proteases, glucanohydrolases, endoglycosidases, amylases, mutanases, lipases and mucinases or compatible mixtures thereof. In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present compositions are disclosed in U.S. Pat. No. 5,000,939 to Dring et al., U.S. Pat. Nos. 4,992,420; 4,355,022; 4,154,815; 4,058,595; 3,991,177; and 3,696,191 all incorporated herein by reference. An enzyme of a mixture of several compatible enzymes in the current invention constitutes 0.002% to 2.0% in one embodiment or 0.05% to 1.5% in another embodiment or in yet another embodiment 0.1% to 0.5%.

Water may be present in the oral compositions. Water, employed in the preparation of commercial oral compositions should be deionized and free of organic impurities. Water commonly makes up the balance of the compositions and in certain aspects of any of the oral care compositions includes: 5% to 45%, e.g., 10% to 20%, e.g., 25-35%, by weight of the oral compositions. This amount of water includes the free water which is added plus that amount which is introduced with other materials such as with sorbitol or silica or any components disclosed herein.

In certain aspects the oral care compositions of the disclosure, comprise a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

Oral Rinse

An oral rinse formulation comprising one or more of the compound(s) disclosed herein and optionally a pharmaceutically acceptable carrier is provided. The formulation may include an additive for stability and/or a flavoring. The oral rinse composition may be formulated to be of sufficient strength that the quantity that a person can conveniently hold in the mouth at one time is adequate for one home care treatment, and treatment need not be carried out more frequently than every six hours, by inserting in the mouth a quantity of the rinse, suitably 2 to 10 milliliters, holding it in the mouth for a sufficient time, suitably one to two minutes, and removing the rinse, as by spitting out and such amounts of saliva as have accumulated in the mouth under the stimulating effect of the rinse. When measured with standard medicine droppers, the quantity of the rinse may be suitably 50 to 250 drops, e.g., 50 to 70 drops.

The oral rinse may contain additional compounds such as to increase stability, enhance treatment of oral mucositis, and kill germs that cause plaque and gingivitis, and/or improve taste. These additional compounds may comprise one or more additives, buffering agents, preservatives, flavorings, chelating agents, anti-oxidants, humectants, stabilizers (including antioxidants), colorants, and other additives used in preparations administered into the oral cavity. Additional compounds could also include corticosteroids (e.g., dexamethasone), anti-histamines (e.g., diphenylhydramine), topical anesthetics (e.g., lidocaine), or anti-fungal agents (e.g., nystatin).

In some aspects, the oral rinse in accordance with the present disclosure further comprises water and pharmaceutically acceptable excipients or additives such as one or more oils (e.g., an oil selected from the group comprising anethole, anisole, camphor, methyl salicylate, vanillin, eugenol, furaneol, linalool, menthol, thymol, cinnamaldehyde, citral, methyl butanoate, pentylbutanoate, pentylpentanoate, tea tree oil, peppermint oil, spearmint oil, pineapplemint oil and eucalyptus oil), sweetening agents (e.g., sorbitol), thickening agents (e.g., xanthan gum, carrageenan, carbomer, or HPMC (hydroxypropyl methyl cellulose)), preservative agents (e.g., sodium benzoate, methyl paraben, or propyl paraben), water, emulsifiers (e.g., polysorbate 80 (or Tween™ 80)), and/or at least one antacid (e.g., aluminum or magnesium hydroxide). It will be appreciated by persons skilled in the art that the above list of excipients and/or additives is provided merely by way of example and that various other such components may be used in the formulation of the present disclosure.

The oral rinses may have a pH of 3 to 8, such as a pH of 4 to 6.5. A preparation having a pH of less than about 3 would be likely to cause a stinging sensation. Furthermore, the preparations having a higher pH are often unpleasant to use. The preparations may be buffered as necessary to provide the appropriate pH. Appropriate buffer systems may include citrate, acetate, tromethamine and benzoate systems. However, any buffer system commonly used for preparing medicinal compositions would be appropriate. While the vehicle used generally is primarily water, other vehicles may be present such as alcohols, glycols (polyethylene glycol or polypropylene glycol are examples), glycerin, and the like may be used to solubilize the compound(s). Surfactants may include anionic, nonionic, amphoteric and cationic surfactants, which are known in the art as appropriate ingredients for oral rinses. Procedures for choosing the optimum pH and buffering agents are well known. Other factors that affect stability in solution are also well known. For example, antioxidants may be added to reduce the rate of degradation Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, and gellan gums. Gellan gums may be employed as viscosity modifying agents since aqueous solutions containing certain gellan gums may be prepared so that they will experience an increase in viscosity upon contact with electrolytes. Saliva contains electrolytes that can interact with such a gellan containing solution so as to increase their viscosity.

Flavorings that may be comprised within the oral rinse may include peppermint, citrus flavorings, berry flavorings, vanilla, cinnamon, and sweeteners, either natural or artificial. Flavorings that are known to increase salivary electrolyte concentrations may be added to increase the magnitude of the viscosity change. The increased viscosity may promote retention of the solutions in the oral cavity and provide greater effectiveness due to increased contact time with the affected tissues.

In some aspects, antimicrobial preservatives may be components of the oral rinse formulation in cases where it is necessary to inhibit microbial growth. Suitable preservatives include, but are not limited to the alkyl parabens, benzoic acid, and benzyl alcohol. The quantity of preservative may be determined by conducting standard antimicrobial preservative effectiveness tests such as that described in the United States Pharmacopoeia.

In further embodiments, the oral rinse may comprise one or more antibiotics. In one embodiment, the composition is administered orally as a fluid. The fluid can be, for example, a solution, a suspension, a paste, or a gel. In some embodiments, the fluid is held in the mouth for a recommended period of time before being discharged from the mouth.

Methods of using the formulations disclosed herein generally involve applying the formulations topically to mucosal surfaces of the oral cavity. In some aspects, the method comprises one to six applications per day. The typical volume of the oral rinse may be between 5-15 ml.

Further embodiments of the present disclosure provide pharmaceutical compositions comprising the oral rinse and a pharmaceutically acceptable excipient.

The composition may also include conventional additives such as adhesive agents, antioxidants, crosslinking or curing agents, pH regulators, pigments, dyes, refractive particles, conductive species, antimicrobial agents, active agents and permeation enhancers. In those embodiments wherein adhesion is to be reduced or eliminated, conventional detackifying agents may also be used. These additives, and amounts thereof, are selected in such a way that they do not significantly interfere with the desired chemical and physical properties of the composition.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” or “pharmaceutically acceptable excipient” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

Non-limiting examples of suitable excipients, diluents, and carriers include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; carriers such as propylene glycol and ethyl alcohol, and lubricants such as talc, calcium and magnesium stearate, and solid polyethyl glycols.

Additives may be present in the compositions, such as flavoring, sweetening or coloring agents, or preservatives. Mint, such as from peppermint or spearmint, cinnamon, eucalyptus, citrus, cassia, anise and menthol are examples of suitable flavoring agents. Flavoring agents may be present in the oral compositions in an amount in the range of from 0 to 3%; up to 2%, such as up to 0.5%, or around 0.2%, in the case of liquid compositions. Sweeteners include artificial or natural sweetening agents, such as sodium saccharin, sucrose, glucose, saccharin, dextrose, levulose, lactose, mannitol, sorbitol, fructose, maltose, xylitol, thaumatin, aspartame, D-tryptophan, dihydrochalcones, acesulfame, and any combinations thereof, which may be present in an amount in the range of from 0 to 2%, up to 1% w/w, such as 0.05 to 0.3% w/w of the oral composition.

Other optional ingredients of oral aqueous compositions include humectants, surfactants (non-ionic, cationic or amphoteric), thickeners, gums and binding agents. A humectant adds body to the oral rinse formulation and retains moisture in a dentifrice composition. In addition, a humectant helps to prevent microbial deterioration during storage of the formulation. It also assists in maintaining phase stability and provides a way to formulate a transparent or translucent dentifrice. Suitable humectants include glycerin, xylitol, glycerol and glycols such as propylene glycol, which may be present in an amount of up to 50% w/w each, but total humectant in one embodiment may not more than about 60-80% w/w of the composition. For example, liquid compositions may comprise up to about 30% glycerine plus up to about 5%, e.g., about 2% w/w xylitol.

When the oral compositions are in the form of a mouth spray, in one embodiment a film forming agent may be included at up to about 3% w/w of the oral composition, such as in the range of from 0 to 0.1%, about 0.001 to 0.01%, such as about 0.005% w/w of the oral composition. Suitable film-formers include (in addition to sodium hyaluronate) those sold under the tradename

The oral rinse composition may be used topically to the mucosal tissue in the oral cavity every 6 hours. It will be appreciated that this dosing regimen is merely exemplary, and the dosing schedule can be varied according to each individual, to the severity of oral mucositis and in accord with other parameters. By way of example, the e oral rinse formulation can be applied topically to mucosal surfaces of the oral cavity, in some embodiments, one to five applications per day, and may continue s. By way of another example, the oral rinse formulation can be administered to the desired local area, one, two, three, four, five or more times per day.

In one embodiment, the method includes administering to a patient one or more of the identified compound(s) in a solution or suspension. The solution or suspension is administered as, for example, a mouth-rinse. In another embodiment, the method includes administering a solid dosage form to the oral cavity of a patient. The solid dosage form is one intended to be retained in the oral cavity and not necessarily ingested or swallowed by the patient.

Treatment according to the disclosed methods can be 1-2 days or up to 1 week and then maintained, for example, until any symptoms have substantially cleared or the risk of developing such symptoms has passed. In other examples, treatment is maintained for 1-4 or 2-3 weeks. Treatment can be carried out at intervals determined to be appropriate by those of skill in the art. For example, the administration can be carried out 1, 2, 3, 4 or more times/day.

### Subjects

The subject may be any animal, including a human and non-human animals. Non-human animals include all vertebrates, e.g., mammals and non-mammals, such as non-human primates, sheep, dogs, cats, cows, horses, chickens, amphibians, and reptiles, or mammals, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

The invention will be described by the following non-limiting examples.

### Example 1

### Methods

Sg were cultured in a total of 40 mL DMEM for 24 hrs. After 200 μL of Sg suspension reached 0.2 at OD600 value in a 96-well plate, the Sg metabolites were collected by centrifugation at 3000 g for 10 min and then filtered through a 0.22 μm filter.

### Results

The commensal species Streptococcus gordonii (Sg) serves as an early colonizer in the dental plaque biofilm and regulates the pathogenesis of periodontal pathogens. It was reported recently that Sg reduced Porphyromonas gingivalis (Pg) invasion and modulated the inflammation in oral epithelial cells. However, the potential roles of Sg metabolites in periodontal inflammation remains unknown.

To explore the function of Sg metabolites in the homeostasis of periodontal bacteria and inflammatory modulation of host cells, Sg metabolites were collected from the Sg (ATCC 33399) supernatant cultured in DMEM. The metabolites function on proliferation and colonization of periodontal bacteria was tested, including Sg, Streptococcus sanguinis (Ss, ATCC 10556), Streptococcus mitis (Sm, ATCC 49456), Streptococcus oralis (So, ATCC 35037), Pg (ATCC 33277), Tannerella forsythia (Tf, ATCC 43037), and Treponema denticola (Td, ATCC 35405). The toxicities of the compounds were tested as well as their function on inflammatory regulation of human macrophages, epithelial cells, and gingival fibroblasts under Pg derived lipopolysaccharide (Pg-LPS) challenge.

Sg metabolites significantly promoted the proliferation of Sg and So, and reduced the proliferation of Pg, Tf and Td, and the biofilm formation of Pg. Sg metabolites were non-cytotoxic. Treatment with Sg metabolites significantly modulated proinflammatory cytokines, including IL-1β, 6, and 8, induced by Pg-LPS challenge.

Thus, Sg metabolites may provide for the treatment and prevention of periodontitis by maintaining microbiome symbiosis and modulating the immune responses of host cells.

### Example 2

Sg are commensal bacteria that help maintain microbiome symbiosis that acts as a barrier to ascension of pathogenic bacteria. To develop a safe and efficient therapeutic tool for periodontitis that not only modulates inflammatory responses to pathogen invasion but also defends against oral microbiome dysbiosis, the molecular function(s) of Streptococcus gordonii (Sg) metabolites in regulating periodontal inflammation and oral microbiome homeostasis were explored. It was found that Sg metabolites can be applied for periodontitis prevention and treatment.

It was found that Sg prevented the invasion of Porphyromonas gingivalis (Pg), a key pathogenic bacterium of periodontitis, into oral epithelial cells. Sg also reprogrammed the cells to resist Pg induced Zeb2, a transcriptional factor that regulates inflammation. Sg metabolites significantly promoted commensal bacteria proliferation, including Sg, S. sanguinis (Ss), and S. oralis (So), and inhibited the proliferation of pathogen bacteria, including Pg and Treponema denticola (Td). The metabolites also downregulated proinflammatory cytokines induced by Pg metabolites and lipopolysaccharide (Pg-LPS). The Sg metabolites significantly upregulated anti-inflammatory microRNAs (miR), including miR-200c that has been demonstrated to potently prevent periodontitis in an animal model. Thus, Sg metabolites attenuate periodontitis by modulating inflammatory responses of oral host cells and preventing oral microbiome dysbiosis. By identifying bioactive components and determining their function against periodontitis, compounds useful to prevent and treat periodontitis are identified.

Determine the Preventive Function of Sg Metabolites on Periodontitis In Vivo.

In vitro studies have revealed that Sg metabolites effectively downregulated IL-1β, 6, and 8 under Pg challenge. Sg metabolites may mitigate the periodontal inflammation and alveolar bone loss in a mouse model of periodontitis. A mouse periodontitis model is employed with a ligature and Pg inoculation to determine the effectiveness of administering metabolites in attenuating periodontitis in vivo. The μCT, histomorphometric and immunohistochemical analyses are used to determine periodontal inflammation and alveolar bone loss. The ability of Sg metabolites to reduce Pg proliferation and colonialization in vivo is determined.

Identify bioactive components among Sg metabolites that mitigate inflammatory responses. UPLC analysis identified that L-Norleucine has the highest concentration of negative charges among Sg metabolites. L-Norleucine effectively downregulated IL-6 and IL-8 in macrophages. Thus, L-Norleucine is a bioactive Sg metabolite that regulates the inflammatory response of host cells and balances bacterial homeostasis. The function of L-Norleucine on mitigating the inflammation of host cells is determined using human oral epithelial cells, gingival fibroblasts, and macrophages under Pg-LPS challenge. Periodontitis associated inflammatory cytokines and osteoclastogenic mediators are quantified using qRT-PCR and ELISA. RNA-seq is employed to determine the molecular functions of L-Norleucine in regulating inflammation. The function of L-Norleucine on oral bacterial homeostasis, including proliferation and colonization of commensal and periodontopathogenic species, is determined.

Dysbiosis of the oral microbiome initiates periodontitis. While oral anaerobic bacteria, including Porphyromonas gingivalis (Pg), Treponema denticola (Td), and Tannerella forsythia (Tf), were traditionally considered as causative agents of periodontitis due to their virulence properties, advanced findings have revealed that a more diverse periodontitis-associated microbiota is involved in the disease etiology (Darveau, 2010; Socransky & Haffajee, 2005). Specifically, in the transition from periodontal health to periodontitis, a dramatic shift from a symbiotic microbial community composed mostly of facultative bacteria to a dysbiotic microbial community structure composed mainly of anaerobic bacteria has been identified (Bartold & Van Dyke, 2013). Virulence factors that are enriched within the dysbiotic oral microbiota are adapted to increase in inflammatory environments like the subgingival crevice (Hajishengallis et al., 2012; Abusleme et al., 2013; Perez-Chaparro et al., 2014). The polymicrobial synergy among dysbiotic species perturbs the ecologically balanced biofilm associated with periodontal tissue homeostasis and facilitates the shift towards disease-associated microbial species. The dysbiosis eventually becomes extensive enough to comprise a pathogenic entity that induces periodontitis in oral tissues of susceptible individuals (Hajishengallis, 2015). Therefore, maintaining a healthy balance within the subgingival microbiome and preserving the symbiotic microbial community against a dysbiotic shift can help prevent the development of periodontitis.

An exaggerated host inflammatory response contributes to the progression of periodontitis. Accumulated evidence has begun to suggest that the microbial dysbiosis may only initiate disease in the context of other risk factors that are associated with host genotype, stress, diet or risk-related behavior such as smoking. The host immune response against a dysbiotic microbiome plays a key role in periodontitis progression. In host cells, Toll-like receptors (TLRs) recognize periodontal pathogens and trigger the up-regulation of IL-1β, 6, and TNF-α to resist the infection (Darveau, 2010; Hajishengallis et al., 2012; Di Benedetto et al., 2013). TLR-mediated signaling pathways also lead to activation of NF-κB (Herath et al., 2013). These cytokines and transcription factors in turn further amplify the inflammatory response and lead to the production of matrix metalloproteinases and stimulate the production of chemokines (Darveau, 2010; Hajishengallis et al., 2012; Di Benedetto et al., 2013; Herath et al., 2013; Graves et al., 2011). Major proinflammatory molecules and transcription factors including INF-α, IL-1β, 6, 8, 12, 18, NF-kB, and RANKL are up-regulated in resident cells including dendritic, epithelial, and gingival cells, osteoblasts, and periodontal ligament fibroblasts. Compounding the response, migrating cells, including lymphocytes and phagocytes, produce RANKL, TNF-α, and IL-17 (Darveau, 2010; Di Benedetto et al., 2013). Eventually, a cascade of events leads to activation of osteoclasts and subsequent bone resorption via the RANKL-OPG axis (Cochran, 2008; Mogi et al., 2004; Crotti et al., 2003). A poorly controlled host immune response has been postulated to generate a self-perpetuating pathogenic cycle where dysbiosis and inflammation reinforce each other by forming a positive feedback loop in periodontitis. Additionally, the prolonged proinflammatory activities also impair bone formation by reducing differentiation of osteoblasts and their progenitor cells (Yang et al., 2013; Lacey et al., 2009; Hikiji et al., 2000; Wang et al., 2012; Chang et al., 2013; Chang et al., 2009). Therefore, while bacterially derived factors initiate periodontitis, its perpetuation or progression occurs mainly as a result of activating host-derived immune and inflammatory defense mechanisms. microRNAs mediate the inflammation of periodontitis. MicroRNAs (miRs) are small non-coding RNAs that promote the degradation of, and/or repress the translation of, mRNA through sequence specific interactions with specific mRNA targets. miRs actively participate in the progression and management of the inflammatory response, including during the onset and development of periodontitis. Specifically, miRs are significantly differentially expressed between a healthy state and periodontitis (Xie et al., 2011; Saito et al., 2017). miRs have emerged as important transcriptional regulators that target inflammation-related mediators, including TNF-α, IL-1, IL-6, and IL-8 (Nahid et al., 2011; Hong et al., 2016a; Du et al., 2016; Yue et al., 201&). Thus, manipulating anti-inflammatory miRs to modulate inflammation could be used to treat periodontitis in addition to the compound(s) disclosed herein For example, miR-200c were significantly reduced in the gingival tissues of periodontitis patients perhaps via Pg-LPS-induced upregulation of Zeb1 (Stoecklin-Wasmer et al., 2012; Naqvi et al., 2016; Sztukowska et al., 2016). miR-200c directly targets the 3′ UTRs of IL-6/8, Ifrd1, and CCL-5, and down-regulates these proinflammatory and osteoclastogenic mediators in human periodontal ligament and gingival fibroblasts (Hong et al., 2016b; Akkouch et al., 2019). Local application of miR-200c can effectively suppress chronic inflammation and alveolar bone loss in rodent models of periodontitis by targeting and downregulating IL-6, 8, Ifrd1, and NF-kB (Akkouch et al., 2019). miR-146a is also negative feedback to IL-1β and TNF-α, and Hey2 Lina et al., 2019). In the mouse model of periodontitis, miR-146a mimic exhibited protective function on periodontitis associated bone loss (Jiang et al., 2018).

Specific commensal bacterial metabolites may be used to prevent periodontitis. Traditionally commensal organisms in polymicrobial communities can antagonize the action of pathogens through colonization resistance (Darveau, 2010; Abranches et al., 2018; Khan et al., 2019). They can engage in antimicrobial activities by producing bacteriocins or compete for niches and nutrients. In addition, commensals regulate basic developmental features and functions of the immune system that balances a vigorous defense against overt pathogens while maintaining tolerance to innocuous antigens (Belkaid & Harrison, 2017). The commensal microbiome can also induce homeostatic immunity that couples antimicrobial function with tissue repair. Streptococcus gordonii (Sg), a Gram-positive bacterium, is a commensal species that is commonly found in the skin, oral cavity, and intestine. Although Sg can be an opportunistic pathogen that may cause local or systemic diseases under specific circumstances (Park et al., 2020), accumulated evidence suggests that Sg may modulate interactions between the bacterial community and the host by regulating signaling pathways in host epithelial cells. Specifically, recent studies have demonstrated that Sg can reprogram epithelial cell global transcriptional patterns following Pg-induced gingival epithelial cell proliferation, highlighting the potential for Sg to be used in periodontitis prevention and treatment Mans et al., 2009). Sg effectively prevented the invasion of Pg into oral epithelial cells and reprogrammed the cells to resist Pg-induced Zeb2, a transcriptional factor that regulates inflammation (Hanel et al., 2020; Ohshima et al., 2019). Our preliminary studies have revealed that Sg metabolites significantly promoted the proliferation of commensal bacteria and inhibited the proliferation and colonization of periodontopathogenic bacteria. The Sg metabolites also effectively downregulated IL-1β, 6, and 8 induced by Pg. In addition, Sg metabolites effectively increased anti-inflammatory miRs including miR-200c. Therefore, Sg metabolites may represent a tool for the treatment and prevention of periodontitis by maintaining microbiome symbiosis and modulating the immune responses of host cells.

### Results

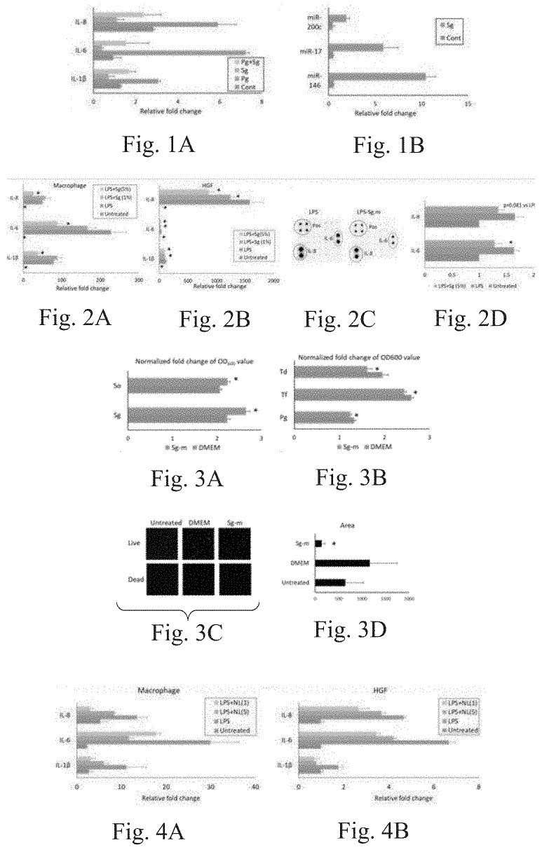

Sg supernatant downregulates IL-1β, 6 and 8 induced by Pg supernatant in macrophages and upregulates miR-200c, 17, 29a, and 146. Both Sg (ATCC 33399) and Pg (ATCC 33277) were cultured in BHI at 37° C. (Camargo et al., 2021; Soory, 1995). Sg were incubated in 5% CO₂, while Pg were incubated in 90% N₂, 5% H₂, 5% CO₂. After the densities of the Sg and Pg were adjusted to the same value at OD₆₀₀, the supernatants were collected after centrifugation at 3000 g for 10 min and then filtered using a 0.22 μm filter. 5% v/v Sg or Pg supernatant was added to mouse macrophages (RAW 264.7) in 24-well plates (2×10⁴ cells/per well) in DMEM medium for 24 hrs. Uninoculated BHI was used as a control. As expected, Pg supernatant increased the transcripts of IL-1β, 6, and 8, whereas Sg supernatant effectively reduced the proinflammatory cytokines, compared to the control. The Sg supernatant also mitigated the cytokine induction by Pg (FIG. 1A). Additionally, the Sg supernatant downregulated anti-inflammatory miRs, including miR-17,146, and 200c (FIG. 1B) (Liu et al., 2011; Li et al., 2018).

Sg metabolites modulate proinflammatory cytokines in human periodontal associated cells in vitro. Sg were cultured in a total of 40 ml DMEM for 24 hrs. After 200 μl of Sg suspension reached 0.2 at OD₆₀₀ value in a 96-well plate, the Sg metabolites were collected by centrifugation at 3000 g for 10 min and then filtered through a 0.22 μm filter. No toxicity of Sg metabolites was detected using an MTT assay on primary human bone marrow mesenchymal stromal cells (BMSCs) and primary human gingival fibroblasts (HGF) (data no shown). The modulation of Sg metabolites on inflammatory cytokines was tested using human monocyte-derived macrophages (MDM) and HGF. The MDM were generated by inducing a human macrophage cell line (THP-1) using PMA at 10 ng/ml. The MDM at 2×10⁶ cells/per well and HGF at 2×10⁴ cells/per well were cultured in 24-well plates using DMEM with 10% FBS and 1% PS. The Sg metabolites at 1 and 5% v/v were added and incubated for 24 hrs before challenging with Pg-LPS at 100 ng/ml. Pg-LPS significantly increased the transcripts of IL-1β, 6, and 8 in MDM and HGF. However, Sg metabolites significantly downregulated the transcripts of the cytokines (FIG. 2A, B). In addition, Sg metabolites significantly downregulated the IL-6 protein in the HGF after Pg-LPS challenge measured using the Human Inflammation Array C1 (RayBio® C-Series) (FIG. 2C, D).

Sg metabolites promote commensal bacterial proliferation and inhibit periodontopathogenic bacterial proliferation and colonization. Commensal bacteria, Sg, S. sanguinis (Ss, ATCC10556), S. mitis (Sm, ATCC49456) and S. oralis (So, ATCC 35037) were cultured in BHI medium. In addition, pathogenic bacteria, Pg was cultured using BHI and Tf (ATCC43037), and Td (ATCC 35405) were grown in Thioglycollate medium with vitamin K and Hemin in 90% N2, 5% H2, 5% CO2. The Sg metabolites were diluted using DMEM and added to the bacteria with the similar OD₆₀₀ value. The same amount of DMEM was used as a control. During the exponential growth of bacteria, the OD₆₀₀ value was measured to determine the proliferation. Treatment with Sg metabolites at 5% v/v significantly increased the proliferation of Sg and So (FIG. 3A). Sg metabolites also significantly reduced the yield of Pg, Tf; and Td after 24 hrs (FIG. 3B). In addition, to determine the function of Sg metabolites on Pg colonization, Pg were inoculated in 24-well plates containing BHI or BHI supplemented with DMEM or Sg metabolites. The plates were incubated in an anaerobic chamber for 48 hrs. With a Live/Dead staining kit and histomorphometrical analysis under a fluorescent microscope, we observed that the DMEM control promoted Pg attachment (or early biofilm formation), whereas Sg metabolites significantly inhibited the Pg attachment (FIG. 3C, D).

### CONCLUSION

While Sg has been recognized as potentially contributing to the maintenance of a healthy oral microbiome and modulating the immune responses of host cells, the mechanisms by which it does so have not been thoroughly investigated. This application seeks to do so in the context of a highly prevalent oral disease—periodontitis. This disclosure provides for the effectiveness of Sg metabolites on prevention and treatment of periodontitis. In particular, the effectiveness of Sg metabolites on attenuating inflammation in a mouse model of periodontitis and protecting the healthy balance of the oral microbiome from periodontopathogenic invasion was confirmed. Also, the function of L-Norleucine in regulating inflammation and its effects on oral microbiomes was explored. The identified bioactive components among Sg metabolites may also be applied for other inflammation related diseases, including pulpitis and temporomandibular joint osteoarthritis.

### Determine the Preventive Function of Sg Metabolites on Periodontitis In Vivo.

Preliminary in vitro studies revealed that Sg metabolites could effectively modulate proinflammatory cytokines of human macrophages and gingival fibroblasts after Pg-LPS challenge. In addition, the Sg metabolites effectively promoted oral commensal bacterial proliferation and inhibited periodontopathogenic bacteria proliferation and biofilm formation. This evidence supports the hypothesis that Sg metabolites may be of benefit for periodontitis treatment and prevention. To validate the therapeutic effectiveness of Sg metabolites, the function of Sg metabolites in mitigating periodontal inflammation and alveolar bone loss and preventing periodontopathogenic bacterial colonization are determined using animal models of periodontitis. The Baker mouse model of periodontitis is well established for analyzing alveolar bone resorption and periodontal microbiome variations induced by oral bacterial inoculums (Yamada et al., 2018; Genco et al., 1991; Ishida et al., 2017). However, the alveolar bone resorption is relatively small, and the time to induce a significant bone loss is relatively long in this model (5-7 weeks). Instead, a ligature model of periodontitis can induce more severe and extensive alveolar bone loss within 1-2 weeks and has been extensively used to determine the therapeutic effectiveness of pharmaceutic tools on periodontal inflammation (Marchesan et al., 2018). Thus, both models are used to determine Sg metabolites' function in preventing oral dysbiotic bacteria and mitigating periodontal inflammation and alveolar bone resorption. For in vivo studies, male and female mice are used in equal proportions. There are sex differences in metabolic homeostasis and immune responses (Chang et al., 2018; Varghese et al., 2017; Mauvais-Jarvis, 2017; Zore et al., 2018). Therefore, the data from males and females is separately analyzed.

Determine the inhibitory function of Sg metabolites towards periodontopathogenic bacteria-induced periodontitis in vivo. The Baker mouse model of periodontitis is established as follows. Briefly, sulfamethoxazole (700 μg/ml) and trimethoprim (400 μg/ml) are provided in drinking water to 4-5-week-old pathogen-free mice BALB/c mice for 10 days before the Pg oral infection. A total of 80 mice (40 male and 40 female) are then divided into TEN groups receiving different treatments: 1) sham controls; 2) untreated+Pg; 3) Sg (1%)+Pg; 4) Sg (1%) alone; 5) DMEM (1%)+Pg; 6) DMEM (1%) alone; 7) Sg (5%)+Pg; 8) Sg (5%) alone; 9) DMEM (5%)+Pg; and 10) DMEM (5%) alone. The mice are given the Sg metabolites at 1 and 5% v/v or DMEM at the same concentrations in drinking water after the antibiotics. The Sg metabolites are prepared as described herein. For oral bacterial inoculation, Pg (ATCC 33277) are cultured and collected as described above. 10⁹ CFU of live Pg (ATCC 33277) in 100 μl of PBS with 2.5% carboxymethylcellulose will be applied three times at 2-day intervals to the gingival margin of each mouse under brief isoflurane anesthesia. Mice receiving 100 μl of PBS with 2.5% carboxymethylcellulose serve as controls. After inoculation with Pg, the mice fast for 1 hr. The mice are weighed weekly. Oral swab samples are taken 2, 3, and 4 weeks after the infection phase for DNA extraction. The mice are sacrificed 4 weeks after the oral Pg challenge, and the maxillary specimens and serum samples from mice with different treatments are collected as in previous studies (Krongbaramee et al., 2021). The transcript and protein levels of proinflammatory cytokines and mediators are quantified, including IL-6, IL-8, Ifrd1, NF-kB p65/p50, MYD88, IL-1β, TNF-α, IKK-α/β, and RANKL, using real-time PCR and ELISA in gingival tissues and blood. Anti-inflammatory cytokines, including IL-4, 10, 13, 19, and IL-35, are also measured as well as periodontitis related miRs. The alveolar bone loss is quantitated using μCT, including measuring the distances from the cementoenamel junction (CEJ) to the alveolar bone crest (ABC), bone volume (BV), tissue volume (TV), bone volume/tissue volume ratio (BV/TV), and bone mineral density (BMD) within the interdental region. Histomorphometric analysis in a double-blind manner is performed after H&E and TRAP staining. In addition, total DNA is extracted from oral swabs using the QIAamp DNA Mini Kit and colonization of Pg in the oral cavity assessed using specific primers for the bacterium's 16S rRNA gene as described in other studies (Tran & Rudney, 1996).

Outcomes. Mice inoculated with Pg exhibit Pg colonization of gingival tissues. Sg metabolite(s) effectively reduce Pg colonization, whereas DMEM does not have an effect. Alveolar bone resorption is observed in the mice inoculated with Pg. Treatment with Sg metabolite(s) effectively mitigate the bone resorption. Sg metabolite(s) significantly increase levels of anti-inflammatory cytokines and downregulate gingival and serum proinflammatory cytokines and mediators induced by Pg inoculation. Pg inoculation time and the species are variables in the mouse model of periodontitis. Thus, Pg inoculation time or different Pg strains may be varied in order to induce periodontitis accompanied by Pg colonization and alveolar bone resorption. Sg concentration range may also be varied to adjust the efficacy of inhibitory function on alveolar bone loss and Pg colonization.

Determine Sg metabolites' function on attenuating inflammation and alveolar bone loss in a ligature model of periodontitis 12 week-old male and female BALB/c mice are given Sg metabolite(s) at 1 and 5% v/v, or DMEM as a control, in their drinking water for 7 days before the ligature procedure. A total of 48 mice (24 male and 24 female) are divided into SIX groups with different treatments, including: 1) sham controls; 2) untreated+ligature; 3) DMEM (1%)+ligature; 4) DMEM (5%)+ligature; 5) Sg (1%)+ligature; and 6) Sg (5%)+ligature. 5-0 silk suture is used around the 2nd maxillary molars to create the ligature-induced periodontitis. Sg metabolite(s) and DMEM provided in water bottles are continued until euthanasia after 2 weeks. Mice are weighed weekly. Gingival tissue surrounding maxillary molars and serum samples from mice with different treatments are collected. miRs and transcript and protein levels of inflammatory cytokines and mediators are quantified. The alveolar bone loss is quantitated using μCT, including the distances from the CEJ to the ABC in the interdental region between M2 and M3, BV/TV, and BMD within the interdental region. Histomorphometric analysis are performed as described previously (Krongbaramee et al., 2021).

Outcomes. Sg metabolite(s) effectively reduce inflammation of periodontitis induced by ligature and in the Pg in the Baker model. Sg metabolite(s) downregulate gingival and serum proinflammatory cytokines and mediators induced by the ligature and the effect of Pg colonization/biofilm formation. Sg metabolites may also increase levels of anti-inflammatory cytokines. In addition, Sg metabolites reduce alveolar bone loss induced by the ligature. The concentration ranges of Sg metabolite(s) may be adjusted to enhance the inhibitory function on periodontal inflammation and alveolar bone loss.

Identify Bioactive Components Among Sg Metabolites that Mitigate Inflammatory Responses.

To develop an Sg metabolite based application for periodontitis treatment and prevention, identification of the bioactive components among the Sg metabolites, along with an understanding of the underlying molecular mechanism(s) that maintain symbiotic microbiomes and mitigate immune responses, is conducted. Preliminary studies using UPLC identified 1099 components comprising the Sg metabolites. Metabolites present at high concentrations are investigated for their function in mitigating the inflammation of host cells and preventing dysbiotic oral microbiomes, alone and when compared to the collective activity of Sg metabolites.

L-Norleucine as well as other metabolites modulate proinflammatory cytokines in macrophages and HGF. L-Norleucine was obtained commercially (Sigma) and dissolved in DMEM. Mouse macrophages and human HGF were cultured with DMEM supplemented with different concentrations of L-Norleucine and subsequently challenged with Pg-LPS at 100 ng/ml. While Pg-LPS upregulated transcripts of IL-1β, 6, and 8, treatment with L-Norleucine effectively downregulated the transcripts of the proinflammatory cytokines in macrophages and HGFs (FIG. 4). These data strongly indicate that L-Norleucine may potentially modulate host inflammatory responses in periodontitis.

### Research Design and Procedures

Determine the function of L-Norleucine in relation to collective Sg metabolite components on the inflammatory response and the oral microbiome. The function of L-Norleucine and other metabolites at a concentration that matches its representation within Sg metabolite preparations are tested on inflammatory responses using periodontitis associated cells. The concentration of Sg metabolites is measured using UPLC. Human gingival epithelial cells (hGECs, Axolbio), human macrophages, and HGF are treated with the Sg metabolites (1 and 5% v/v) or a proportional concentration thereof and challenged with Pg-LPS (100 ng/ml). The proinflammatory cytokines and mediators and anti-inflammatory cytokines and miRs are quantitatively measured after 12, 24, and 48 hrs. To determine regulation on commensal and periodontopathogenic bacteria proliferation and colonization, the commensal species, including Sg, Streptococcus sanguinis (Ss), Streptococcus mitis (Sm), and Streptococcus oralis (So), and pathogenic species, including Pg, Tannerella forsythia (Tf), and Treponema denticola (Td), are cultured as described herein. Sg metabolites (1, 5, and 10% v/v) and the same L-Norleucine doses are added to the bacterial cultures. The proliferation rate of bacteria and the colonization of Pg is measured.

Outcomes. Sg metabolites administration results in modulation of inflammation and regulation on commensal and pathogenic bacterial proliferation and colonization. Certain Sg metabolites may exhibit more potent activity than others or when combined with other Sg metabolites.

Determine the molecular function of metabolite(s) on periodontal inflammation. mRNA-seq and bioinformatics analyses are used to determine the molecular function of Sg metabolites on inflammation and osteoclastogenesis. Human macrophages and hGECs are treated with Sg metabolite(s), e.g., at the same concentration, for 24 hrs. Non-treated cells serve as controls. Total RNA is collected, and the mRNA-seq data analysis workflow includes the following steps: (i) reads that pass quality control are mapped to the genome by STAR; (ii) featureCounts is used to estimate transcript expression levels; (iii) Deseq2 is used to determine differential expression. The differentially expressed genes are confirmed using real-time PCR.

Outcomes. RNA-seq analysis reveals the signaling pathways modulated by Sg metabolite(s) when affecting inflammation and osteoclastogenesis in human macrophages and hGECs. This clarifies at least one of the underlying mechanisms by which Sg metabolites can promote periodontitis treatment and prevention.

Statistical Analysis: A sample size of 8 (per group/condition) is used to test Sg metabolites' function on periodontitis in the Baker and ligature mouse models. However, the final required sample sizes are estimated based upon pilot study data using a type I error of alpha=0.05 and 80% power. Longitudinal repeated measure analysis methodology, including ANOVA with repeated measures and linear mixed models with random effects, is used for analyzing in vivo studies. The post-hoc adjustments for multiple comparisons of the effects of metabolites on proinflammatory and osteoclastogenic mediators is conducted using the Tukey and Holm methods.

The experiments clarify the bioactive potential of Sg metabolites, and components therein, as a treatment or preventive for periodontitis via an ability to modulate inflammation and preserve a symbiotic oral microbiome.

### Example 3

### HCA, an Sg SCS Metabolite, Modulates Proinflammatory Cytokines in Cellular and Organismic Levels.

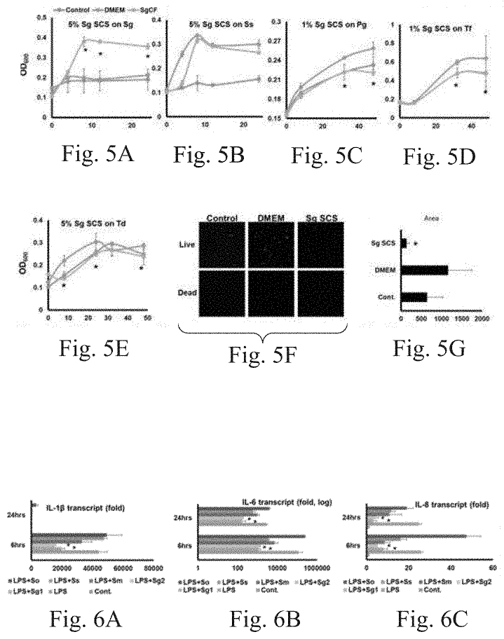

Human monocyte-derived macrophages (MDM) using THP-1 cells and HGF were cultured in a 24-well plate with DMEM and challenged with Pg-LPS at 100 ng/ml. Treatment of HCA at 0.1 and 1.0 μM significantly downregulated transcripts of IL-1β, 6, and 8 in HGF and THP-1 cells after 6 hrs (FIGS. 5A and C).

HCA at 0.1 μM also significantly reduced protein levels of IL-6 and IL-8 in the supernatant of THP-1 and HGF lysate after 24 hrs (FIGS. 5B and D). In addition, freshly isolated epidydimal white adipose tissues (eWAT) from mice on a HFD for 16 weeks were minced and cultured in 24-well plates and treated with HCA at 0.5 and 1.0 μM in DMEM/F-12 containing 10% cosmic calf serum (CCS) and 1% penicillin-streptomycin (basal growth media, BGM). At 16 hours of post treatment, transcripts of IL-6, IL-1β, and Nos2 were significantly reduced in WAT treated with HCA (FIG. 5E). The protein level of IL-6 in cultured media are significantly reduced (FIG. 5F). These preliminary data are strong evidence that Sg metabolites have the potential to modulate host inflammatory responses and attenuate inflammation.

HCA Inhibits Pg Proliferation and Upregulates miR-200c.

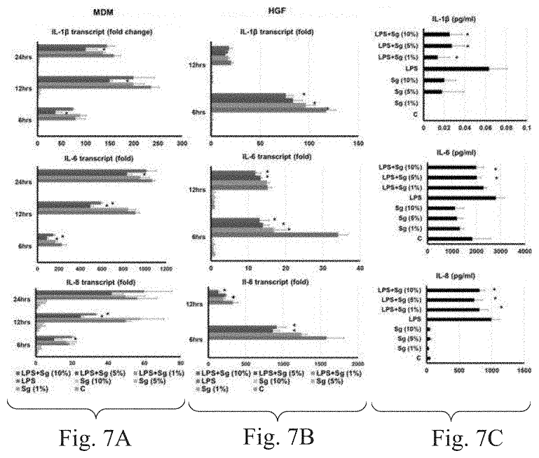

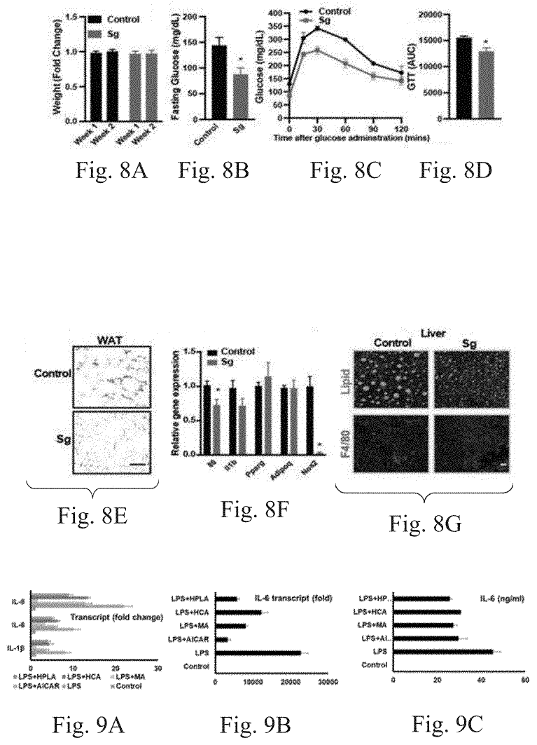

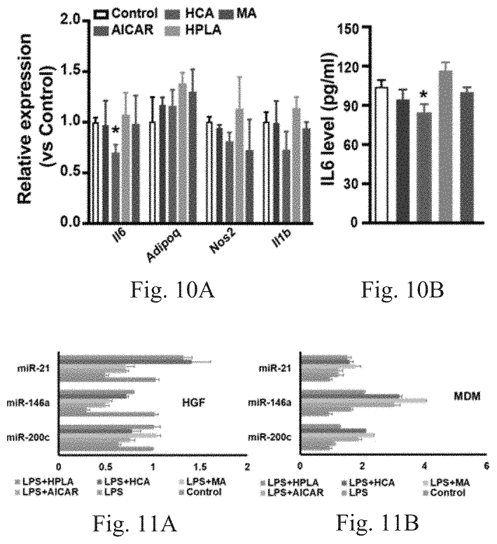

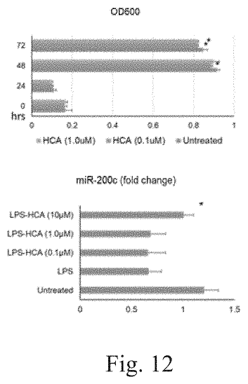

Pg (ATCC 49417), a key pathogen of periodontitis, was cultured at 24 well-plates with BHI medium at 90% N₂, 5% H₂, 5% CO₂. HCA at 0.1 and 10 μM was applied. FIG. 6A summarizes that HCA treatment significantly reduced OD₆₀₀ value after 48 and 72 hrs compared to untreated controls (All data are presented as means±SEM. *: p<0.05 vs untreated, ANOVA) (FIG. 6)

HCA Upregulates miR-200c after Pg-LPS Challenge.

Human MDM was cultured in a 24-well plate and treated with HCA at different concentrations and Pg-LPS at 100 ng/mi for 24 hrs. While Pg-LPS reduced miR-200c, treatment of HCA at 10 μM effectively restored the downregulation and significantly upregulated miR-200c expression (FIG. 7). (All data are presented as means±SEM. *: p<0.05 vs untreated, ANOVA).

### Example 4

Obesity, one of the most serious health concerns worldwide, is a risk factor for several debilitating diseases including diabetes and cardiovascular disease (PMID. 29021283) and although lifestyle changes, including exercise and caloric restriction have been shown to be effective for short-term weight loss, obesity persists due to weight regain. Therefore, alternative strategies are needed for sustainable weight management.

Dietary modification such as structured lipid use (e.g., triacylglycerols and fatty acids (FA)) has long been associated with beneficial outcomes for diabetic and obese patients (PM ID. 11880549; 35011045; 34684300; 26652763; 18326600; 12775120). For instance, unsaturated fatty acids (UFAs), found within diet and serum, are protective against nonalcoholic fatty liver disease (PMID. 21856859; 12324287). Further, dietary-mediated polyunsaturated fatty acids (PUFAs) display anti-obesogenic and -atherosclerotic effects (PMID. 29174025, 30754681). Finally, branched fatty acyl esters of hydroxyl fatty acids (FAHFAs, including short-, medium- and long-chain fatty acids), have been shown to contain antidiabetic properties (PMID. 29566292; 27080715). Ultimately, it is critical to understand the complex regulatory roles, functions and implications of FA consumption on human health and disease.

Hydroxycarboxylic medium chain fatty acids (HCMCFAs), such as lactate and ketone bodies metabolized from coconut oil and dairy products, are important substrates and/or intermediates of energy metabolism (PMID. 27080715). Characterized by a chain length of 6-12 carbons, physiochemically, HCMCFAs are water-soluble in the intestinal lumen and cytoplasm of target cells; anatomically, HCMCFAs are absorbed predominantly via the portal vein into the liver bypassing the lymphatic system (PMID. 20655716). Physiologically, HCMCFAs have been shown to modulate immune cell function (PMID. 29375572), contain antioxidant potential (PMID. 35262212), reduce inflammatory responses (PMID. 26799523; 33207743; 12480795) and activate ligand-dependent transcription factors involved in insulin sensitivity (i.e., PPARgamma, PMID. 22649490; 34831163). Further, in humans and rodents, in vivo studies have associated MCFAs with increased oxidative metabolism and reduced diet-induced adiposity (PMID. 24078708; 11880549; 12634436; 21872431; 34836064). Moreover, in vitro studies have shown MCFAs increase mitochondrial oxidative capacity while reducing cell-associated lipid concentration, oxidative stress and monolayer permeability (PMID. 24078708; 27080715; 29991957).

However, although important for human health, HCMCFA-mediated effects on diet induced obesity (DIO) are incompletely understood. In this study, the potential protective effects and underlying mechanisms of a medium chain, omega-hydroxy fatty acid, 6-hydroxycaproic/carboxylic acid (6-HCA), on diet-induced obesity (DIO) was investigated using mouse and cell culture models. Herein is evidence that HCA improves DIO-mediated inflammation and insulin resistance in DIO mice while decreasing FFA release and pro-inflammatory mediators from white adipocytes. These findings demonstrate the potential for novel FAs in management of diet-induced obesity and associated comorbidities.

### Methods

### Cell Culture

3T3-L1 (CL-173-ATCC, PMID. 26451286) were grown in DMEM containing 10% fetal bovine serum (FBS) and 1% pen/strep. Cells were induced to differentiate 2 d after reaching confluence by supplementing growth media with 3 nM insulin (Humulin R, Lily, Humulin R U-500, 0002-8501-01), 0.25 nM dexamethasone (Sigma, D4902), 2 uM rosiglitazone (Sigma, R2408) and 0.5 mM 1-methyl-3-isobutyl-xanthine (Sigma, I5879). From day 3 until day 7, cells were maintained in growth media supplemented with 3 nM insulin after which the mature adipocytes were maintained in growth media. Cells were then incubated for 24 hr in the presence or absence of 10 ng/ml TNF (PeproTech, 315-01A) with or without 0.1 uM 6-Hydroxycaproic acid (HCA, Sigma, 515302).

### Animal Experiments

Animal care and experimental procedures were performed with approval from the University of Iowa's Institutional Animal Care and Use Committee. Animals received humane care in compliance with the Guide for the Care and Use of Laboratory Animals (National Academies Press, 2011) and with the Principles of Laboratory Animal Care formulated by the National Society for Medical Research. C57BL/6J mice (The Jackson Laboratory, 000664) were kept on a 12-hour light/dark cycle. Mice used to generate the DIO model were placed on a 60% kCal high-fat diet (HFD, Research Diets, D12492) immediately after weaning (i.e., at 3 weeks of age). After 16 weeks, lean and DIO-mice were intraperitoneally (IP) injected with 0.5 uM HCA or PBS control every other day for three weeks. Body weight was measured weekly. All tissues were harvested, frozen in liquid nitrogen, and kept at −80° C. until processed.

### Quantitative Real-Time RT-PCR

Total RNA was isolated using TRIzol reagent (Invitrogen, 15-596-018) and reverse transcribed into cDNA using the iScript cDNA synthesis kit (Bio-Rad, 1708890). Quantitative real-time RT-PCR analysis was performed using SYBR Green (Invitrogen, KCQS00).

### Western Blot Analysis

Proteins were extracted from cells or tissues and subjected to SDS-polyacrylamide gel electrophoresis, as previously described (Yang et al., 2015). Membranes were incubated with anti-p-AKT (Ser473, Cell Signaling, 9271); anti-AKT1 (H-136, Santa Cruz, sc-8312) or anti-ACTB (H-300, Santa Cruz, sc-10731) at 1:1000 and then incubated with the appropriate secondary antibody conjugated with horseradish peroxidase (1:5000, Santa Cruz, sc-2005 or 1:5000, Cell Signaling Technology, 7074S). Signal was detected using the ChemiDoc Touch Imaging System (Bio-Rad), and densitometric analyses of western blot images were performed using Image Lab software (Bio-Rad).

### Immunohistochemistry and Immunofluorescence

For immunohistochemistry, tissues were fixed with 4% PFA and sectioned at 5 μm thick, followed by deparaffinization and rehydration processes. Tissue sections were stained using H&E. The images were observed under a Nikon microscope (10×).

### Biochemical Analysis and Cytokine Measurements

Blood samples were centrifuged (4° C., 5000 g, 30 min) to obtain serum. Ten microliters of serum per sample were required for each index analysis. Serum alanine aminotransferase (ALT) and aspartate aminotransferase (AST) were measured by commercial kits. Serum IL-1β, IL-6 and free fatty acid (FFA) content were measured by ELISA (Il-1beta, Biolegend, Cat No. 432604; Il6) or a FFA fluorometric kit (Cayman Chemical, No. 700310), respectively, and then normalized to total protein (BCA).

### Metabolic Phenotyping

Whole-body energy expenditure and body composition: Respiratory exchange ratio (RER) and locomotor activity were monitored using a Comprehensive Lab Animal Monitoring System (CLAMS, Columbus Instruments) at the Fraternal Order of Eagles Metabolic Phenotypic Core. Body composition was measured by using Bruker Minispecs (LF50).

### Insulin Tolerance Test

Animals were fasted for 6 hours prior to ITT. Insulin tolerance was tested by measuring glucose concentration at different time points after intraperitoneal (IP) insulin injection (0.5 U/kg body weight; Humulin R) (Qian et al., 2018).

### Statistical Analysis

Results are expressed as the mean t the standard error of the mean (SEM); n represents the number of individual mice (biological replicates) or individual experiments (technical replicates) as indicated in the figure legends. We performed the Shapiro-Wilk Normality test in experiments that have a relatively large sample size (n>5) and found that these data pass the normality test (alpha=0.05). Data were further analyzed with two-tailed Student's and Welch's t-test for two-group comparisons or ANOVA for multiple comparisons. For both One-Way ANOVA and Two-Way ANOVA, Tukey's post-hoc multiple comparisons were applied as recommended by Prism. In all cases, GraphPad Prism (GraphPad Software Prism 8) was used for the calculations.

### Results

### 6-Hydroxycaproic Acid (HCA) Reduced HFD-Mediated Weight Gain and Improved Hyperlipidemia

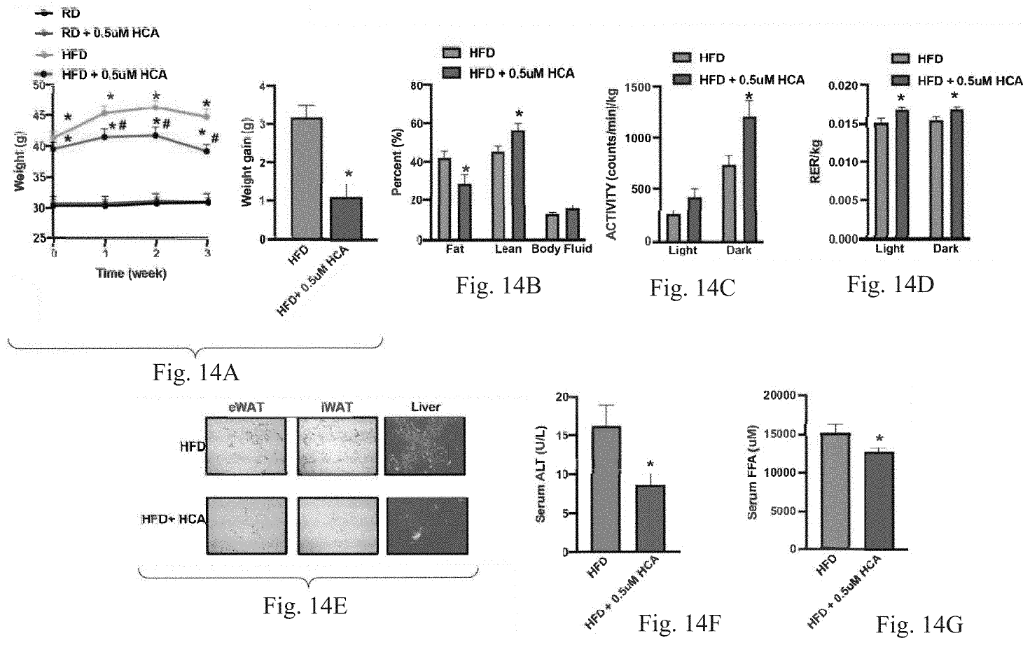

To determine the effects of HCA on HFD-fed mice, body weight (BW) and composition were measured. Compared to RD, mice fed a HFD gained more BW (FIG. 14A). However, compared with the HFD group, BW gain was significantly reduced, while lean mass increased, in HCA-treated animals (FIGS. 14A&B). This was associated with increased activity and a shift in respiratory exchange ratio (RER) towards increased carbohydrate metabolism suggesting better fuel utilization (FIGS. 14C&D).

Morphologically, HFD-mediated, HCA-treated liver, inguinal and epididymal white adipose tissue (iWAT and eWAT, respectively) showed improvement compared to HFD-treated animals alone (FIG. 14E). Notably, HFD-mediated serum ALT levels were reduced by HCA treatment demonstrating over nutrition-mediated hepatic toxicity was blunted by HCA exposure (FIG. 14F).

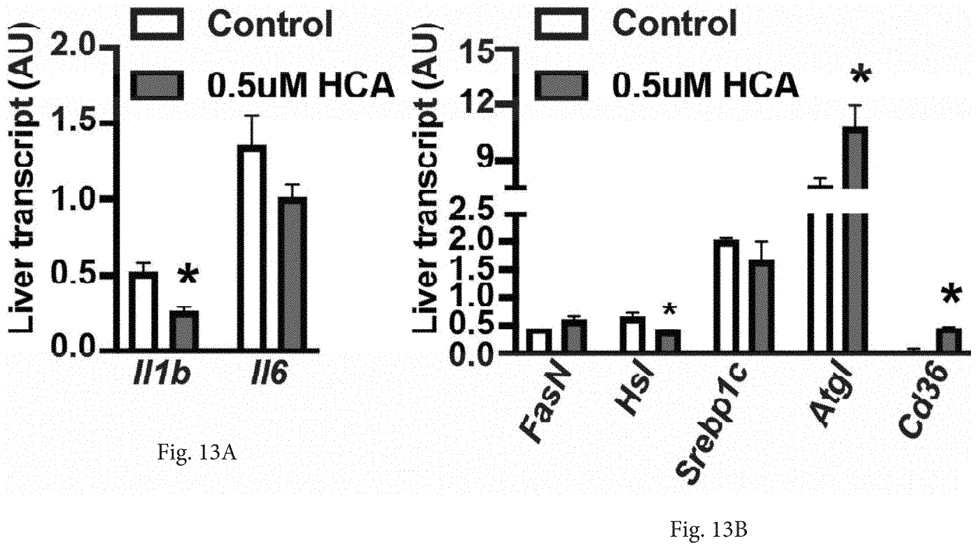

Serum free fatty acid (FFA) content was measured and it was found that HCA treatment reduced circulating levels compared to HFD controls (FIG. 14G). Additionally, liver transcripts involved in lipogenesis and lipolysis were altered-compared to HFD alone, specifically, HCA increased expression of Atg1 and Cd36; in contrast, expression of Hsl was significantly reduced (FIG. 13A). These data indicate HCA modifies hepatic lipolytic activity in DIO mice.

### HCA Improved HFD-Mediated Systemic Glucose Homeostasis and Insulin Sensitivity

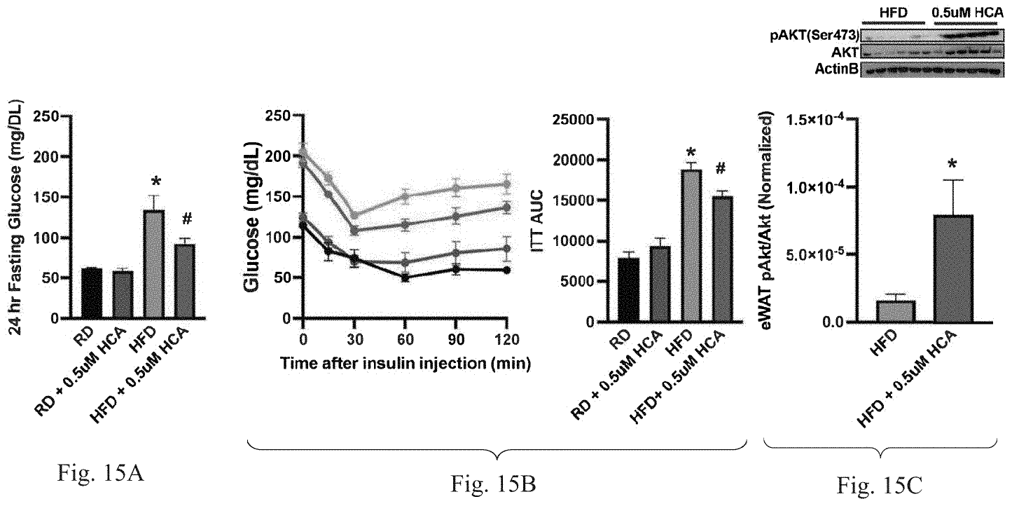

HFD mediated obesity is associated with impaired systemic glucose homeostasis. It was determined whether HCA modulates hyperglycemia and insulin sensitivity. Compared to RD mice, fasting glucose in HFD-fed animals was significantly increased; furthermore, insulin tolerance was impaired (FIGS. 15A&B). While lean HCA-treated mice showed similar results to RD-fed mice, HFD-mediated effects were reduced. Moreover, assessment of insulin signaling in eWAT of HFD-fed mice showed increased pAKT in HCA-treated mice, suggesting improved insulin sensitivity (FIG. 15C).

Modulation of HFD-Mediated Epididymal White Adipose Tissue (eWAT) Transcriptome by HCA

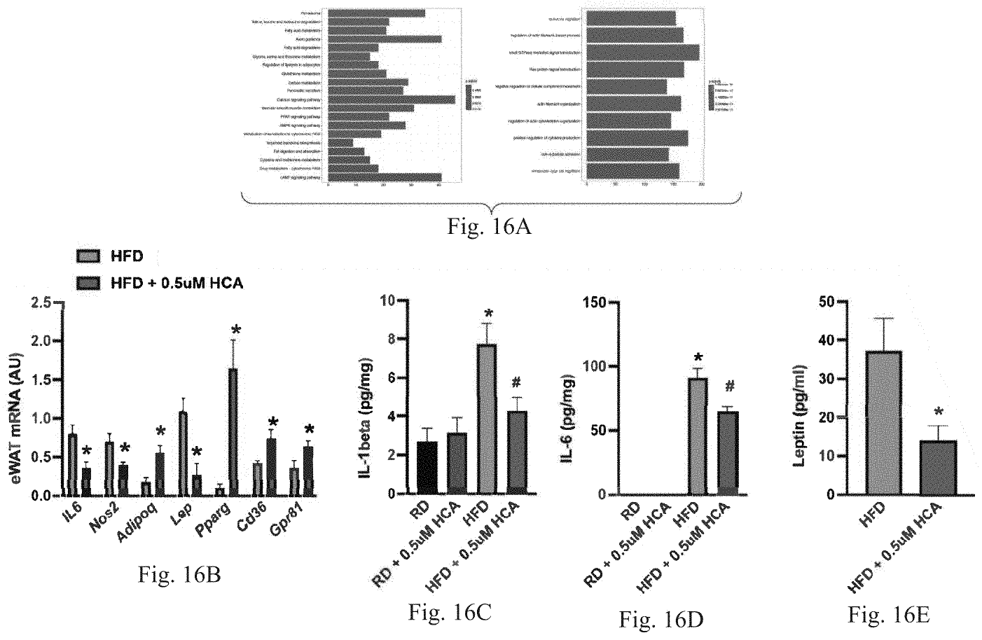

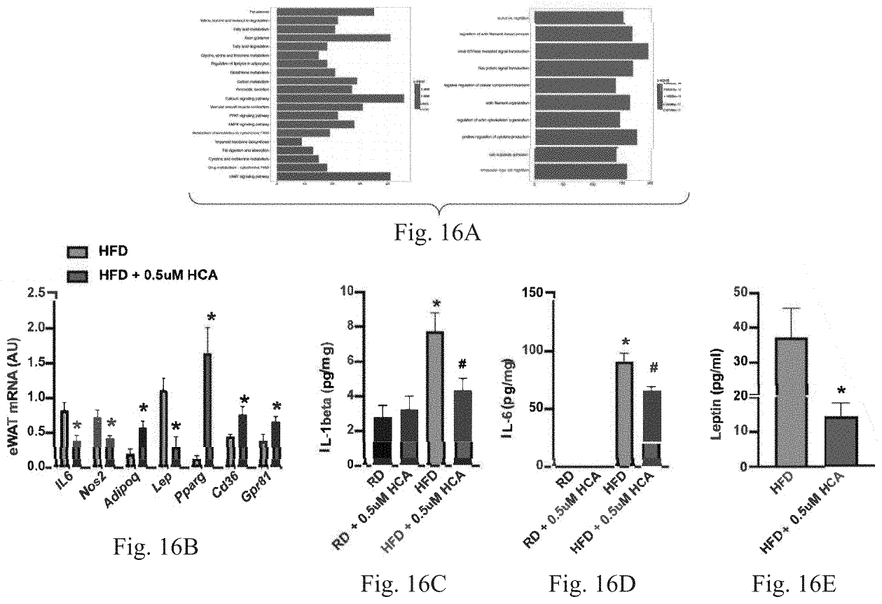

To investigate the HFD-mediated genome-wide changes in RNA levels in the eWAT mediated by HCA, we performed RNA-seq (FIG. 16A). Signaling pathway involved in fatty acid metabolism, oxidation and absorption, PPAR gamma as well as AMPK signaling were significantly increased while chemokine signaling and leukocyte migration were downregulated (FIG. 16A).

### HCA Reduced HFD-Induced Inflammation

The expression of several genes associated with inflammation and WAT-marker expression in eWAT and liver, respectively, was analyzed. In the eWAT of HCA-treated animals, HFD-induced pro-inflammatory (Il6, Nos2) as well as white adipose marker (Lep) mRNA expression were significantly reduced; in contrast, Adipoq and Ppargamma levels increased (FIG. 16B). Notably Cd36 and Gpr81, receptors involved in FA uptake, were also induced compared to HFD controls (FIG. 16B). With regards to the liver, HFD-induced Il1b transcript levels were significantly reduced by HCA (FIG. 13). Changes in mRNA expression were concomitant with reduced serum IL-6, IL-1β, and leptin (FIG. 16C-E).

### HCA Reduced Cytokine-Induced Inflammation in Differentiated Adipocytes

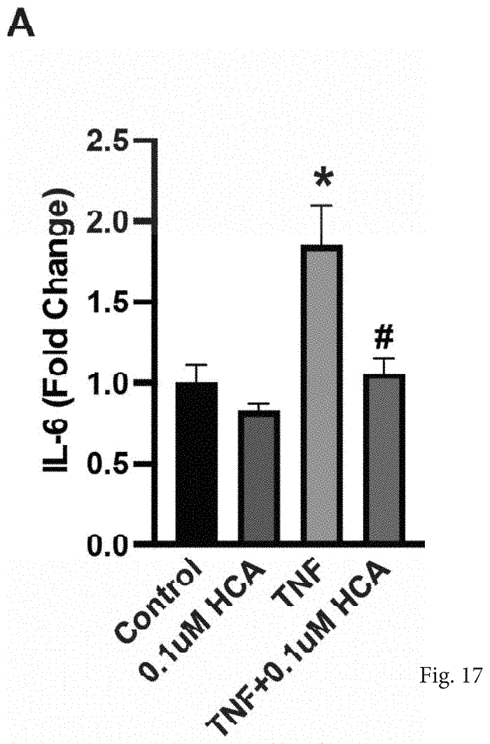

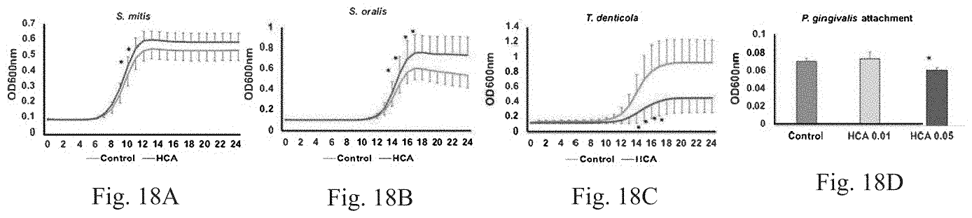

To determine the effects of HCA on adipose-mediated inflammation, 3T3-L1 cells were differentiated into white adipocytes and then exposed to the pro-inflammatory cytokine tumor necrosis factor alpha (TNFa). Here we found TNF-induced IL-6 secretion was significantly reduced by HCA (FIG. 17A).

### Discussion

Although experimental studies suggest dietary HCMCFAs contribute to weight loss by inducing thermogenesis and fat oxidation while reducing adiposity, effects on glucose homeostasis in human patients remain contradictory. For instance, in lean men, dietary intake of MCFA increased serum insulin while reducing glucose levels (PMID. 2187945) or had no effect (PMID. 31869355). In patients with non-insulin-dependent diabetes mellitus (NIDDM), dietary intake of MCFA had no effect or improved insulin-mediated glucose clearance (PMID. 7706596; 1568535; 31869355).

### Example 5

Nearly 50% of American adults have periodontitis, a set of inflammatory diseases that not only cause tooth loss but can also affect systemic health by increasing the risk for many diseases. While periodontitis is considered to have a complex etiology acting at multiple levels, the molecular mechanisms underlying the etiology and pathogenesis of periodontitis remain to be fully unraveled. Oral hygiene, scaling and cleaning, and antibiotics have achieved relative success in arresting the progression of early stage periodontitis that is without systemic disease association; however, surgical intervention is needed for advanced periodontitis. The success rate of the current surgical treatment for moderate to advanced periodontitis is only 50%⁹. Thus, effective tools and strategies that improve prevention and therapy outcomes are needed.

An exaggerated host inflammatory response is a key factor in the initiation and progression of periodontitis. Although specific anaerobic bacterial species have been traditionally considered as causative agents of periodontitis, a more diverse periodontitis-associated microbiota is now considered to be involved in disease etiology. In the transition from periodontal health to periodontitis, a symbiotic microbial community is dramatically shifted to a dysbiotic microbial community composed mainly of anaerobic bacteria. The polymicrobial synergy among dysbiotic species eventually perturbs the ecologically balanced biofilm associated with periodontal tissue homeostasis and facilitates the shift towards disease-associated microbial species. Nevertheless, accumulated evidence suggests that microbial dysbiosis may only initiate disease in the oral tissues of susceptible individuals in the context of other risk factors associated with host genotype, stress, diet or risk-related behavior such as smoking, obesity and diabetes.
The host immune response against the dysbiotic microbiome also plays a key role in periodontitis progression. In host cells, Toll-like receptors (TLRs) initially recognize periodontal pathogens, trigger the up-regulation of IL-1β, 6, and TNF-α, and lead to activation of NF-κB. These cytokines and transcription factors further amplify the inflammatory response and stimulate the production of matrix metalloproteinases and chemokines. Major proinflammatory molecules and transcription factors, including TNF-α, IL-1β, 6, 8, 12, 18, NF-kB, and RANKL, are upregulated in resident cells. Compounding the response, migrating cells produce RANKL, TNF-α, and IL-17. Eventually, a cascade of events leads to activation of osteoclasts and subsequent bone resorption via the RANKL-OPG axis. Therefore, a poorly controlled host immune response has been postulated to generate a self-perpetuating pathogenic cycle where dysbiosis and inflammation reinforce each other by forming a positive feedback loop in periodontitis.
Oral commensal bacteria maintain the homeostasis of microbiomes and modulate host metabolism and immune system. Commensal organisms are known to antagonize pathogens through colonization resistance in polymicrobial communities. However, not all interactions are solely antimicrobial. For example, interactions between commensal Streptococcus gordonii (S. gordonii) and the periopathogen Porphyromonas gingivalis (P. gingivalis) affect colonization and proliferation in a complex and cascading manner that can have variable effects on virulence.

Commensal bacteria are also known to serve as an interface between host metabolism and the immune system via nutrient- and metabolite-dependent mechanisms. They regulate the immune system's basic developmental features and functions that balance a vigorous defense against overt pathogens while maintaining tolerance to innocuous antigens. The commensal microbiome can also induce homeostatic immunity that couples antimicrobial function with tissue repair⁵. S. gordonii can be an opportunistic pathogen that causes local or systemic diseases under specific circumstances. However, accumulated evidence suggests that S. gordonii may modulate interactions between the bacterial community and the host by regulating signaling pathways in host epithelial cells. Specifically, S. gordonii can reprogram epithelial cell global transcriptional patterns following P. gingivalis-induced gingival epithelial cell proliferation. S. gordonii also effectively prevents the invasion of P. gingivalis into oral epithelial cells and reprograms the cells to resist P. gingivalis-induced Zeb2, a transcriptional factor that regulates inflammation. Studies have revealed that S. gordonii spent culture supernatant (Sg-SCS) significantly inhibits the proliferation of periodontopathogenic bacteria, including of P. gingivalis and T. denticola. Sg-SCS also inhibits the attachment of P. gingivalis. The Sg-SCS effectively downregulates inflammation and proinflammatory cytokines in human macrophages, gingival fibroblasts, and epithelial cells (see details in Shu et al, Journal of Periodontology, 2022). These data indicate that Sg-SCS contains beneficial metabolic components that may represent a tool for the treatment and prevention of periodontitis by maintaining microbiome symbiosis and modulating the immune responses of host cells.

Free fatty acids (FFAs) play critical roles in periodontal inflammation. FFAs are important energy sources for body tissues, are classified based on their carbon atom's tail length, including short-chain fatty acids (SCFA, carbons (C): ≤6), medium-chain fatty acids (MCFA, C:6-12), and long-chain fatty acids (LCFA, C: >12). FFAs also play critical functions in many physiology and pathophysiology regulations, including periodontal inflammation. Specifically, while SCFAs produced by gastrointestinal bacteria modulate the inflammatory response and link between the microbiota and the immune system, SCFAs are considered virulence factors when produced locally in periodontal pockets by periodontitis-associated bacteria. SCFA level is found to increase in gingival crevicular fluid of periodontitis patients and their levels vary according to the periodontitis treatment. Pathogenic bacteria-secreted SCFAs haven been demonstrated to stimulate the transmigration of leucocytes through the epithelial layer and impair the integrity by changing junctional and adhesion protein expression. SCFAs can also induce apoptosis in inflamed human gingival fibroblasts and periodontal destruction. SCFA can further stimulate oxidative stress. Thus, SCFA initiate and perpetuate periodontitis by participating in proinflammatory activities.

Long-chain saturated FFAs function similarly to SCFAs in proinflammatory activities by activating NF-kB signaling. However, long-chain polyunsaturated fatty acids (LC-PUFA), including omega-3 and omega-6 LC-PUFA, are considered as important inflammatory modulators and have a substantial effect in anti-inflammatory processes. The levels of serum LC-PUFA are found to increase in periodontitis patients and can vary by periodontitis treatment. Supplementation of LC-PUFA was considered an adjunction in the management of periodontitis. Omega-3- and omega-PUFA potentially modify the inflammation by reducing oxidation. However, clinical evidence to reduce periodontitis remains controversial.

MCFAs (C6 to C12) are saturated FFAs that are mainly found in coconut oil, palm kernel oil, and dairy products. MCFAs have been demonstrated to possess antimicrobial effects against algae, fungi, protozoa, viruses, and Gram-positive bacteria⁴⁹. MCFA has been shown to potently improve metabolic function in obesity and diabetes through direct receptor-mediated intracellular pathways and altering circulating levels of hormones and metabolites. However, very few studies of MCFAs have been performed in periodontitis except a clinical study from Buduneli's group. In this study, the level of 3-OH—C12 in periodontitis patients' saliva is significantly lower than healthy controls. The level of the MCFA are further reduced in periodontitis patients who smoke. This clinical report strongly indicates that MCFA levels in saliva are closely associated with periodontitis. Our recent studies have identified 6-hydroxycaproic acid (HCA), an omega-hydroxy MCFA, as being enriched in Sg-SCS analyzed using Ultra Performance Liquid Chromatography (UPLC). HCA has strong regulatory capacities on the proliferation of commensal and periodontopathogenic bacteria and anti-inflammation in periodontitis-associated cells in vitro and mouse models. HCA, a MCFA, may be treat and prevent periodontitis by maintaining microbiome symbiosis and modulating immunity.

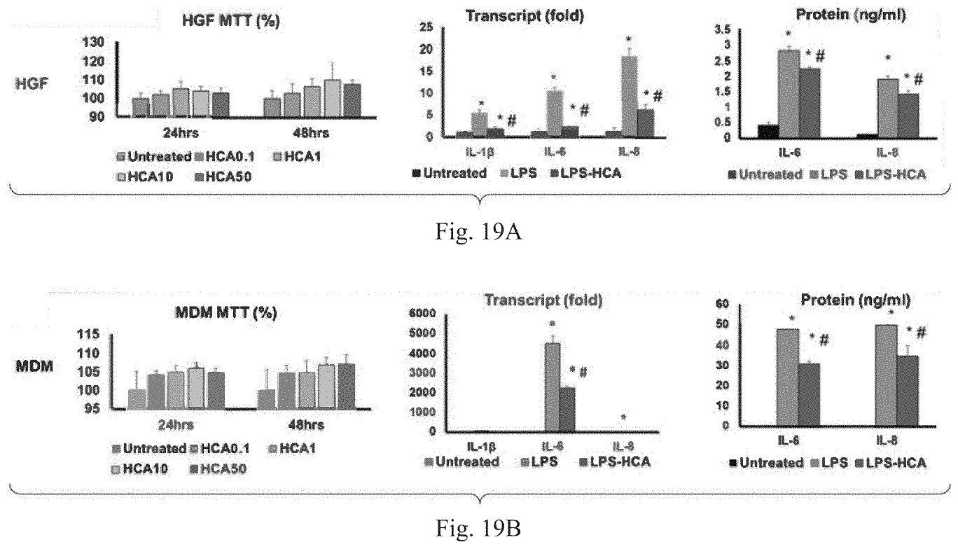

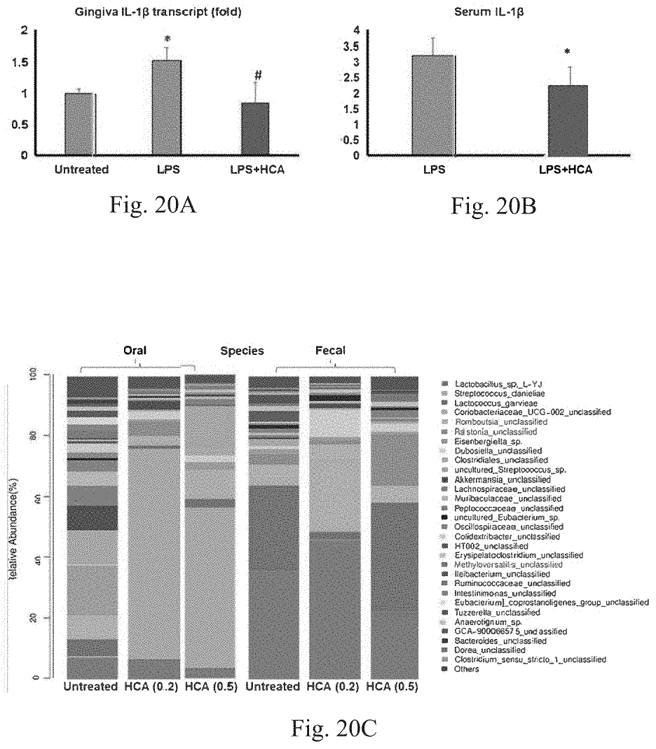

HCA promotes proliferation of health related bacteria and inhibits proliferation and attachment of periodontopathogenic bacteria. Promoting the growth of health-related commensal bacteria while inhibiting proliferation and attachment of pathogenic bacteria may be effective at preventing or treating periodontal inflammation. To determine if HCA has an effect on the growth properties of commensal bacteria and periodontopathogens, HCA at different concentrations was added to aerobic culture of S. mitis and S. oralis and anaerobic culture of T. denticola as described in our published manuscript (see detail in Shu et al, Journal of Periodontology, 2022). FIGS. 18A-C summarize the functions of HCA on the proliferation of specific commensal and pathogenic bacteria. Notably, HCA significantly exhibits capabilities to improve proliferation of S. mitis (FIG. 18A) and S. oralis (FIG. 18B), but significantly inhibits proliferation of T. denticola (FIG. 1C). Furthermore, the function of HCA on P. gingivalis attachment, a key parameter of adhesion and biofilm formation by periodontopathogenic bacteria, was investigated. HCA at 0.05 μM significantly reduced attachment of P. gingivalis compared to the untreated control, while HCA at 0.01 μM had less of an effect (FIG. 18D). Attachment measurements of P. gingivalis were performed based on our published studies (see detail in Shu et al, Journal of Periodontology, 2022). These data indicate that HCA selectively promotes commensal bacterial proliferation and inhibits proliferation and attachment of periodontopathogenic bacteria.
HCA is non-toxic and effectively inhibits proinflammatory cytokines. To test the biocompatibility and anti-inflammatory properties of HCA, human monocyte-derived macrophages (MDM) and gingival fibroblasts (HGF) were prepared as described in our previous studies. The cells were then treated with HCA from 0.1 to 50 μM. No toxicity was observed after treatment with different concentrations of HCA in either cell type using an MTT assay (FIGS. 19A and B, left column). The anti-inflammatory function of HCA on MDM and HGF after exposure to P. gingivalis lipopolysaccharide (PG-LPS) was investigated. Notably, treatment with HCA at 0.1 μM significantly downregulated transcripts of IL-1β, IL-6, and IL-8 in HGF and MDM after 6 hours following PG-LPS challenge at 0.1 μg/mL (FIGS. 19A and B, middle column). HCA also significantly reduced production of IL-6 and IL-8 protein in HGF and MDM after 24 hrs (FIGS. 19A and B, right column). This is evidence that HCA is non-toxic and can effectively inhibit the expression of proinflammatory cytokines.
HCA mitigates inflammation and affects the composition of microbiomes in vivo. Recent studies investigated whether HCA modulates periodontal inflammation using a mouse model. To induce periodontal inflammation, a total of 1 μl Pg-LPS at 10 μg/μl was directly injected twice a week into the interdental region between maxillary molars of C57BL/6J mice using a Hamilton1700 series syringe according to our previously published studies. 100 μl of HCA at 0.5 μM was administrated I.P. three time a week. After 3 weeks, local injection of PG-LPS significantly upregulated IL-1β transcript in gingival tissues measured using qRT-PCR. However, systemic administration of HCA potently attenuated periodontal inflammation by suppressing gingival IL-1β expression (FIG. 20A). In addition, serum IL-1β production was also significantly reduced by HCA administration (FIG. 20B). These data indicate that administration of HCA potently mitigates periodontal and systemic inflammation in a mouse model of periodontitis.
It was also tested whether HCA affects the composition of the microbiome in vivo. For this purpose, high fat diet-induced obese (DIO) mice (The Jackson Laboratory) received I.P, administration of 100 μl of HCA at 0.2 and 0.5 μM three times a week for 3 weeks. DIO mice treated with PBS were used as a control. Oral samples were collected by swabbing the oral cavity with a cotton-tipped applicator. Fecal pellets were collected from the cages as representative of the intestinal microbiome. Microbial DNA was isolated using the Qiagen DNeasy PowerLyzer® PowerSoil® kit. The composition of the microbiomes was determined by amplifying the V3 and V4 regions of the 16S rRNA genes. Raw data in FASTQ format were filtered and denoised using DADA2 (Divisive Amplicon Denoising Algorithm) to generate amplicon sequence variants (ASV) or operational taxonomical units (OTUs) that were then analyzed by QIIME2. HCA had a power effect that was dose dependent. Changes in species abundances were most noticeable in the oral microbiome where treatment with HCA resulted in large increases in the representation of Streptococcus danieliae and other unclassified streptococci. This experiment demonstrated that treatment with HCA may alter the microbiome.

While SCFAs and LCFAs play critical roles in the oral microbiome and the immune responses of host cells in periodontitis development, less is known about the molecular function of MCFAs and their underlying mechanism(s). This study clarifies the bioactivities of HCA, an anti-inflammatory MCFA identified among S. gordonii metabolites, which may allow for both prevention and treatment of periodontitis. The project includes: 1) confirming the effectiveness of HCA on oral microbiomes and on attenuating periodontal inflammation in human cell culture and a mouse model of periodontitis; 2) exploring the potential receptors of HCA in mediating the functions of HCA in periodontal inflammation. T. The bioactive HCA may also be applied for other inflammation-related diseases, including pulpitis and temporomandibular joint osteoarthritis.

### Determine the Molecular Functions of HCA in Mitigating Inflammation in Periodontal Cells and its Effect on the Growth of Health and Disease Related Oral Taxa.

In preliminary studies it was found that HCA is safe and has the potential to reduce expression of proinflammatory cytokines and alter the composition of the oral microbiome. In order to develop HCA as a 1 tool for periodontitis treatment and prevention, confirmation is obtained of its anti-inflammatory properties in human primary epithelial cells, dendritic cells, and macrophages when challenged with pathogenic P. gingivalis and Pg-LPS.

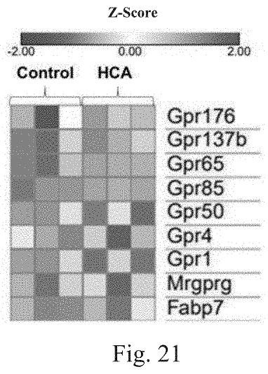

As the physiological sensors of FFAs, the fatty-acid-binding proteins (FABPs) and nuclear lipid-binding protein families, such as peroxisome proliferator activated receptors (PPARs), are known as functional receptors that regulate many physiological and pathophysiological processes of FFAs Some G protein-coupled receptors (GPRs) are also considered as mediating the function of FFAs. The PPARs and GPRs actively participate in periodontitis. Recent studies have also identified upregulation of PPARγ in white adipose tissues (WAT) of obese mice after systemic administration of HCA. An mRNA-seq analysis has also identified upregulation of GPR137B, GPR176, GPR65, GPR85, and FABP7 in WAT of obese mice treated with HCA (FIG. 21). Among these GPRs, GPR137B was reported to involve in IL-4-induced M2 macrophage polarization, and it inhibits osteoclast differentiation and bone resorption. GPR65 also promotes Th1 and Th17 differentiation and inhibits intestinal inflammation. Parallel studies have also identified that PPARα, γ, GPR65, and GPR137B are upregulated in gingival tissues of periodontitis patients (data no shown). FABP7 has been identified to bind unsaturated fatty acids and serve as intracellular transporters for the endocannabinoid anandamide to inhibit inflammation. Protective function of FABP7 from inflammation was observed in mouse autoimmune encephalomyelitis. Thw activities of PPARα and γ, GPR137B, GPR65, and FABP7 under HCA treatment following proinflammatory stimulation are measured. HCA may effectively regulates the nuclear receptors and GPRs in suppressing inflammation.

Additionally, the HCA effects on the proliferation of health- and disease-related representatives from the oral microbiome are investigated. The ability to alter the composition of the microbiome is highly significant as current treatment regimens for chronic inflammatory diseases typically rely on suppressing the total microbial biomass rather than ‘correcting’ the dysbiosis in the microbiome composition. Treatment with HCA clearly presents an opportunity to develop a multi-functional therapeutic that addresses disease-related microbial dysbioses. Efforts to suppress the levels of putative periodontal pathogens and elevate the levels of health-related species may accompany strategies aimed at controlling inflammation. Accordingly, it is desirable to show that HCA kills or suppresses periodontal pathogens while having no effect (or a beneficial effect) on health-related taxa. A panel of disease-related species that have been found by multiple comprehensive studies to be linked to periodontitis: P. gingiwalis, T. denrticola, Tannerella forsythia (T. forsythia), and Aggregatibacter actinomycetemcomitans (A. actinomycetemcomitans) and health-related species, Streptococcus sanguinis (S. sanguinis), Kingella oralis (K. oralis), and Actinomyces oris (A. oris).

Determine the inhibitory function of HCA towards periodontitis-associated inflammatory cytokines. Human periodontal cells, including human dendritic cells (Lonza), oral epithelial cells (Accegen), and human macrophages (ATCC) will be cultured in 24-well plates and treated with HCA at different concentrations (0.1, 1.0, and 10 μM) in the presence or absence of Pg-LPS (100 ng/ml) or pathogenic P. gingivalis (ATCC 49417) at a multiplicity of infection of 100. Transcript and protein levels of periodontitis-associated proinflammatory cytokines and mediators in cell lysates and supernatants, including IL-6, IL-8, Ifrd1, NF-kB p65/p50, MYD88, IL-1β, TNF-α, IKK-α/β and RANKL, and anti-inflammatory cytokines, including IL-4, 10, 13, 19, and IL-35, are measured after 1, 2, 12, 24 and 72 hours using qRT-PCR and ELISA.
Determine the molecular effects of HCA on FFA associated receptors. Transcript and protein levels of PPARγ, PPARα, GPR137B, and GPR65 in human oral epithelial cells and human macrophages after treatment with HCA under P. gingivalis and Pg-LPS challenges are measured. Antagonists of PPAR α and γ (GSK3787 and G3335, Sigma) are used to determine the roles of PPARs when inflammation is suppressed by HCA treatment. GPR137B, GPR65 and FABP7 will be silenced using siRNAs (Santa Cruz Biotechnology) to determine whether they mediate the anti-inflammatory properties of HCA.
Examine the role of metabolites on select oral microbiome representatives. Health-related species, including S. sanguinis, K. oralis, and A. oris will be adjusted to identical (±0.005) OD₆₀₀ values and cultured in 24 well-plates with 500 μl BHI medium. S. sanguinis and A. oris will be cultured aerobically with 95% Air, 5% CO₂ at 37° C., while K. oralis will be incubated in a standard aerobic atmosphere. For the disease-related oral bacteria, P. gingivalis, T. denticola, T. forsythia, and A. actinomycetemcomitans will be adjusted to a standard OD₆₀₀ and cultured in 24 well-plates with 500 μl ml BHI or Thioglycollate medium under anaerobic conditions at 37° C. HCA at 0.1, 1.0, and 10 μM will be tested individually as described in our preliminary studies to determine the effects on growth rate and growth yield.

The data show the anti-inflammatory functions of HCA and its roles on oral bacterial proliferation. Treatment using HCA reduces proinflammatory cytokines and mediators and increase anti-inflammatory cytokines. A selective dose of HCA may effectively reduce more than two proinflammatory cytokine and mediator levels at least 1 to 2-fold compared to controls under conditions of pre-induced inflammation. Additionally, HCA reduces the proliferation of disease-related bacteria and increase that of health-related bacteria.

### Demonstrate the Therapeutic Potential of HCA in Periodontitis.

A previous study has demonstrated that MCFAs are reduced in the saliva of periodontitis patients. HCA, a MCFA, is enriched in anti-inflammatory Sg SCS. There are strong anti-inflammatory capabilities of HCA in human periodontal cells. Systemic administration of HCA can effectively reduce proinflammatory cytokines locally and systemically in a mouse model of periodontitis. HCA may affects microbiome homeostasis and inhibits P. gingivalis attachment. This evidence suggests that HCA may be a therapeutic for periodontitis, e.g., HCA attenuates inflammation and reduces disease-related bacteria in periodontitis.

A mouse model of periodontitis is created by placing a ligature around the maxillary second molar of 8-week-old male and female C57BL/6 mice (The Jackson Laboratory) using P. gingivalis-saturated silk as in previous publications⁶⁷⁻⁶⁹. P. gingivalis-soaked silk ligatures will be prepared by incubating the sterile 6-0 silk ligatures with Schaedler broth containing Pg (wild-type strain, ATCC 49417) for 2 days. The mice are randomly placed in various treatment groups, including: 1) sham controls; 2) ligature without treatment; 3-5) ligature with I.P, administration of 100 μl of HCA at 0.1, 0.5 and 1.0 μM, respectively: 6) ligature with PBS alone. HCA or PBS is injected twice weekly. The mice from different treatment groups will be euthanized at 1 and 2 weeks. To analyze the periodontal inflammation in the mice, qPCR is used to quantify the transcripts of IL-6, IL-8, Ifrd1, NF-kB p65/p50, MYD88, IL-1β, TNF-α, IKK-α/β, and RANKL in gingival surrounding tissues. Anti-inflammatory cytokines, including IL-4, 10, 13, 19, and IL-35 are also measured. The protein levels of the proinflammatory and osteoclastogenic mediators in the lysates of the tissues and blood serum are quantified using a Luminex®-X100 Analyzer (Millipore Corp). The harvested maxillae block sections are fixed in 4% formaldehyde and analyzed using μCT imaging. Periodontal bone resorption, including bone mineral density (BMD) and bone volume/tissue volume (BV/TV) in the alveolar bone of maxillae, the distances between the cemento-enamel junctions (CEJ) to the alveolar bone crest (ABC), are quantified. Hematoxylin and eosin (H&E) and tartrate-resistant acid phosphatase (TRAP) staining and double-blinded histomorphometric analyses are performed to evaluate bone loss and osteoclast activities. In order to determine the function of HCA on inhibiting P. gingivalis proliferation, the bacteria in ligatures with different treatments are collected and the quantity of P. gingivalis are quantified using qPCR and P. gingivalis-specific 16S rRNA gene primers after bacterial genomic DNA is extracted using a QIAamp DNA Mini Kit.

The P. gingivalis-soaked ligature model results in significant bone loss and upregulated proinflammatory cytokines and mediators in periodontal tissues and blood serum. However, injection of HCA effectively reduces proinflammatory cytokines and bone resorption in periodontal tissues and blood serum and reduces P. gingivalis levels. HCA treatment may reduce more than two proinflammatory cytokines and mediators in periodontal tissues at least 0.5-fold compared to controls.

A sample size of 8, and a total of 96 mice (48 male and 48 female), are used to determine the function and underlying mechanism(s) of HCA on minimizing periodontal inflammation. However, sample sizes for may be estimated based upon pilot study data using a type I error of alpha=0.05 and 80/o power. Longitudinal repeated measure analysis methodology, including ANOVA with repeated measures and linear mixed models with random effects, are used for analyzing in vivo studies. The post-hoc adjustments for multiple comparisons of the effects of HCA on proinflammatory and osteoclastogenic mediators are conducted using the Tukey and Holm methods.

In summary, MCFA-based therapeutics for periodontitis are disclosed herein. The molecular function of HCA in mitigating inflammation in human periodontal cells and its effects on the proliferation of health and disease related oral taxa are determined. The function of HCA using an in vivo model of periodontitis is investigated. In addition, the potential receptors that mediate the anti-inflammatory function of HCA, which will provide the cues to understand the molecular mechanism(s) of HCA, are determined.

### REFERENCES

Abranches et al., Microbiol. Spectr., 6: (2018).
Abusleme et al., Isme J., 7:1016 (2013).
Akkouch et al., Stem Cells Dev., 28:1026 (2019).
Bartold & Van Dyke, Periodontol. 2000, 62:203 (2013).
Belkaid & Harrison, Immunity, 46:562 (2017).
Camargo et al., Materials (Basel), 14: (2021).
Chang et al., Curr. Diab. Rep., 18:69 (2018).
Chang et al., Nat. Med., 15:682 (2009).
Chang et al., Proc. Natl. Acad. Sci. USA, 110:9469 (2013).
Cochran, J. Periodontol., 79:1569 (2008).
Crotti et al., J. Periodontal. Res., 38:380 (2003).
D'Aiuto et al., J. Clin. Endocrinol. Metab., 93:3989 (2008)
Darveau, Nat. Rev. Microbiol., 8:481 (2010).
Di Benedetto et al., Clin. Dev. Immunol., 2013:503754 (2013).
Du et al., J. Cell. Mol. Med., 20:1329 (2016).
Eke et al., J. Dent. Res., 91:914 (2012).
Genco et al., Infect. Immun., 59:1255 (1991).
Graves et al., J. Oral. Microbiol., 3: (2011).
Graziani et al., Periodontol. 2000, 75:152 (2017).
Hajishengallis et al., Adv. Exp. Med. Biol., 946:69 (2012).
Hajishengallis, Nat. Rev. Immunol., 15:30 (2015).
Hanel et al., Dent. J. (Basel), 8: (2020).
Herath et al., PLoS One, 8:e58496 (2013).
Hikiji et al., Am. J. Physiol. Endocrinol. Metab., 278:E1031 (2000).
Hong et al., Plos One, 11: (2016a).
Hong et al., PLoS One, 11:e0160915 (2016b).
Ishida et al., NPJ Aging Mech. Dis., 3:15 (2017).
Jiang et al., Biochim. Biophys. Acta Mol. Basis Dis., 1864:925 (2018).
Khan et al., Front. Immunol., 10:1203 (2019).
Kim et al., J. Periodontol., 81: 658 (2010).
Krongbaramee et al., Mol. Ther. Nucleic Acids: (2021).
Lacey et al., Osteoarthritis Cartilage, 17:735 (2009).
Li et al., Cell. Death Dis., 9:480 (2018).
Lin et al., J. Periodontol., 85:779 (2014).
Lina et al., J. Cell. Biochem., 120:645 (2019).
Liu et al., Stem Cells, 29:1804 (2011).
Lundgren et al., J. Clin. Periodontol., 28:23 (2001).
Mans et al., BMC Genomics, 10:380 (2009).
Marchesan et al., Nat. Protoc., 13: 2247 (2018).
Mathur et al., J. Indian. Soc. Periodontol., 15:240 (2011).
Mauvais-Jarvis, Adv. Exp. Med. Biol., 1043:3 (2017).
Mogi et al., J. Dent. Res., 83:166 (2004).
Nahid et al., Infect. Immun., 79:1597 (2011).
Naqvi et al., J. Microb. Biochem. Technol., 8:136 (2016).
Ohshima et al., Proc. Natl. Acad. Sci. USA, 116:8544 (2019).
Olsen, Acta Odontol. Scand., 73:563 (2015).
Park et al., Microorganisms, 8: (2020).
Perez-Chaparro et al., J. Dent. Res., 93:846 (2014).
Pires et al., J. Indian. Soc. Periodontol., 18:14 (2014).
Roberts & Darveau, Periodontol. 2000, 69:18 (2015).
Saito et al., FEBS Open. Bio., 7:981 (2017).
Smiley et al., J. Am. Dent. Assoc., 146:508 (2015).
Socransky & Haffajee, Periodontol. 2000, 38:135 (2005).
Soory, J. Periodontal Res., 30:124 (1995).
Stoecklin-Wasmer et al., J. Dent. Res., 91:934 (2012).
Sztukowska et al., Cell. Microbiol., 18:844 (2016).
Tran & Rudney, J. Clin. Microbiol., 34:2674 (1996).
Varghese et al., Adv. Exp. Med. Biol., 1043:65 (2017).
Wang et al., J. Dent. Res., 21:1003 (2012).
Xie et al., Int. J. Oral. Sci., 3:125 (2011).
Yamada et al., Sci. Rep., 8:9008 (2018).
Yang et al., J. Bone Miner. Res., 28:559 (2013).
Yue et al., J. Endod., 43:1323 (2017).
Zore et al., Mol. Metab., 15:35 (2018).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A composition comprising an amount of one or more agents comprising a hydroxy(C₁₋₁₀)(COOH) or salt thereof, wherein C₁₋₁₀ can be substituted or form a ring, a dicarboxylic acid, a purine nucleoside or analog thereof, a pyrimidine nucleoside or an analog thereof, or an amino acid or analog thereof, effective to inhibit inflammation, and optionally a pharmaceutically acceptable carrier.

2. The composition of claim 1 which comprises one or more of 6-hydroxycaproic acid (HCA), malic acid, 4-hydroxyphenyl lactic acid, acadesine, uridine, or citrulline, or any combination thereof.

3. The composition of claim 1 which is a paste for administration to the teeth or gums.

4. The composition of claim 1 which is a gel.

5. The composition of claim 1 which is suitable for injection.

6. The composition of claim 1 which is suitable for topical application.

7. The composition of claim 1 which is a beverage or a foodstuff.

8. The composition of claim 1 wherein the agent is linked to a targeting molecule.

9. The composition of claim 8 wherein the targeting molecule targets dental plaque.

10. The composition of claim 9 wherein the targeting molecule is chlorhexidine or a salivary mucin.

11. The composition of claim 1 which comprises HCA.

12. The composition of claim 1 which comprises two or more of HCA, malic acid, 4-hydroxyphenyl lactic acid, acadesine, uridine, or citrulline.

13. A method to prevent, inhibit or treat inflammation in a mammal, comprising: administering to the mammal a composition comprising an effective amount of one or more of a hydroxy(C₁₋₁₀)(COOH) or salt thereof), wherein C₁₋₁₀ can be substituted or form a ring, a dicarboxylic acid, a purine nucleoside or analog thereof, a pyrimidine nucleoside or an analog thereof, or an amino acid.

14. The method of claim 13 wherein the composition comprises one or more of 6-hydroxycaproic acid (HCA), mail acid, 4-hydroxyphenyl lactic acid, acadesine, uridine, or citrulline.

15. The method of claim 13 wherein the mammal is a human.

16. The method of claim 13 wherein the mammal has osteoarthritis, is obese, has periodontitis, has gingivitis or has pulpitis.

17. The method of claim 13 wherein the composition is systemically administered, orally administered, locally administered or intra-articularly administered or is administered to the gums.

18. The method of claim 13 wherein the composition is injected.

19. The method of claim 13 wherein the composition is a sustained release formulation.

20. The method of claim 13 wherein the composition is a paste or gel.
